(19) United States
(12) Patent Application Publication
(10) Pub. No.: US 2005/0187409 A1
(43) Pub. Date: Aug. 25, 2005

(54) Inhibitors of RNase P proteins as antibacterial compounds

(76) Inventors: Gordon D. Powers, Malvern, PA (US); Michael A. Sturgess, Quakertown, PA (US)

Correspondence Address: CLARK & ELBING LLP, 101 FEDERAL STREET, BOSTON, MA, 02110, US

(21) Appl. No.: 10/970,190

(22) Filed: Oct. 21, 2004

Related U.S. Application Data
(60) Provisional application No. 60/512,981, filed on Oct. 21, 2003.

(51) Int. Cl. C07C 279/20

(52) U.S. Cl. 564/147

(57) ABSTRACT

The present invention features compounds useful for inhibiting RNase P activity. These compounds can be used as therapeutics for treating or preventing a variety of bacterial infections. The compounds belong to several classes including mono- and bis-guanylhydrazones and benzoic acid compounds.

### CROSS-REFERENCE TO RELATED APPLICATIONS

This application claims benefit of U.S. Provisional Application No. 60/512,981, filed Oct. 21, 2003, which is hereby incorporated by reference.

### BACKGROUND OF THE INVENTION

This invention relates to inhibitors of bacterial ribonuclease P holoenzymes. Such inhibitors are useful as antibacterial agents.

Ribonuclease P (RNase P) is an endoribonuclease that cleaves the 5′-terminal leader sequences of precursor tRNAs. RNase P has been characterized in a representative number of species.

In bacteria, the structure of the RNase P holoenzyme is composed of a catalytic RNA subunit (350-450 nucleotides; encoded by the rnp B gene) and a single protein subunit (110-160 amino acids; encoded by the rnp A gene); both are essential for in vivo activity. In Escherichia coli (E. coli), the RNA subunit is termed M1, and the protein subunit is C5. The C5 protein engages in specific interactions with the M1 RNA to stabilize certain M1 RNA conformations. Through these interactions with M1, C5 plays a critical role in the recognition/binding of some substrates.

Comparison of RNase P protein subunits between bacterial species reveals that their primary structures have only a moderate degree of identity. For example, the protein subunits of Bacillus subtilis (B. subtilis) and E. coli are 30% identical. The functional significance of some conserved amino acid residues has been confirmed by mutagenesis studies that have shown that these conserved amino acids play a significant role in the catalytic function of the RNase P holoenzyme.

The tertiary structure of the RNase P protein subunit expressed in B. subtilis has been determined by X-ray crystallography. The overall topology of α-helices and β-sheets is α1 β1 β2 β3 α2 β4 α3, with an uncommon β3 α2 β4 cross-over connection that may confer specific functional consequences. Another functional aspect of the protein is the long loop connecting β2 to β3, termed the metal binding loop, which binds Zn²⁺ ions and mediates interlattice contacts. In addition, the crystal structure reveals an overall fold that is similar to the ribosomal protein S5, translational elongation factor EF-G (domain IV), and DNA gyrase.

Many pathogens exist for which there are few effective treatments, and the number of strains resistant to available drugs is continually increasing. Thus, improved methods are needed for the treatment and prevention of infections caused by a number of bacteria. Desirably, these treatments kill pathogenic bacteria without harming the tissues of the infected patient.

### SUMMARY OF THE INVENTION

The present invention features compounds useful for inhibiting RNase P activity. These compounds can be used as therapeutics for treating or preventing a variety of bacterial infections.

In one aspect, the invention features a compound of the formula: 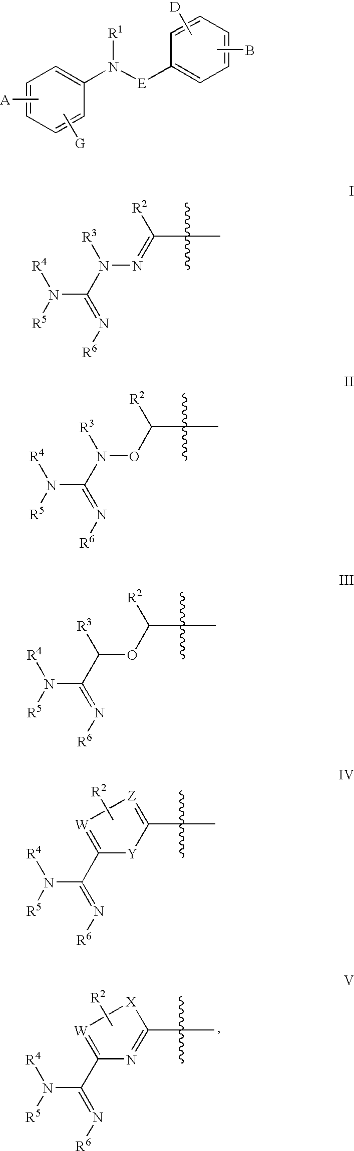
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; E is C═O, C═S, C═CR⁸R⁹, or C═NR⁷; R¹⁻⁹ are independently hydrogen, alkyl, aryl, or aralkyl; W and Z are independently CH, C-alkyl, or N; and X and Y are independently NH, N-alkyl, O, or S. In one embodiment, E is carbonyl. Exemplary compounds of this formula include:

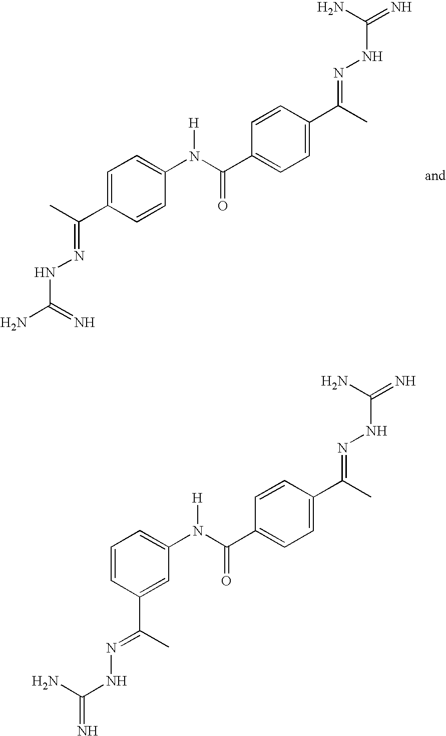

The invention also features a compound of the formula: 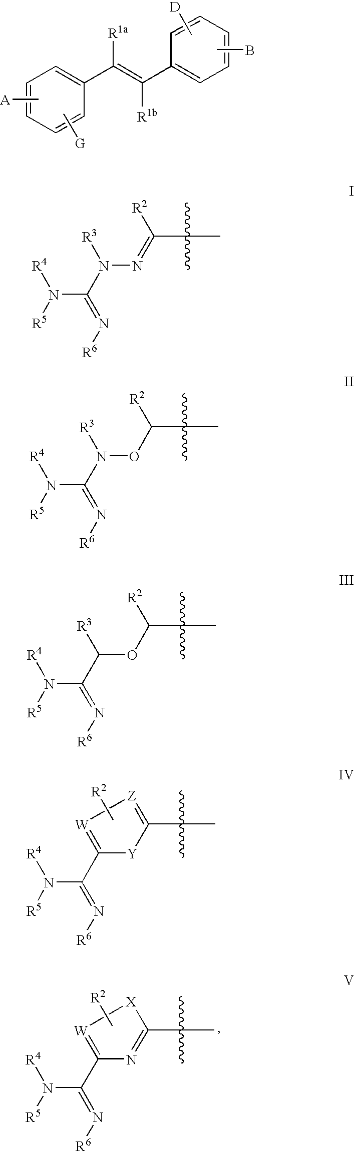
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R^{1a-1b} and R²⁻⁶ are independently hydrogen, alkyl, aryl, or aralkyl; W and Z are independently CH, C-alkyl, or N; and X and Y are independently NH, N-alkyl, O, or S.

The invention further features a compound of the formula: 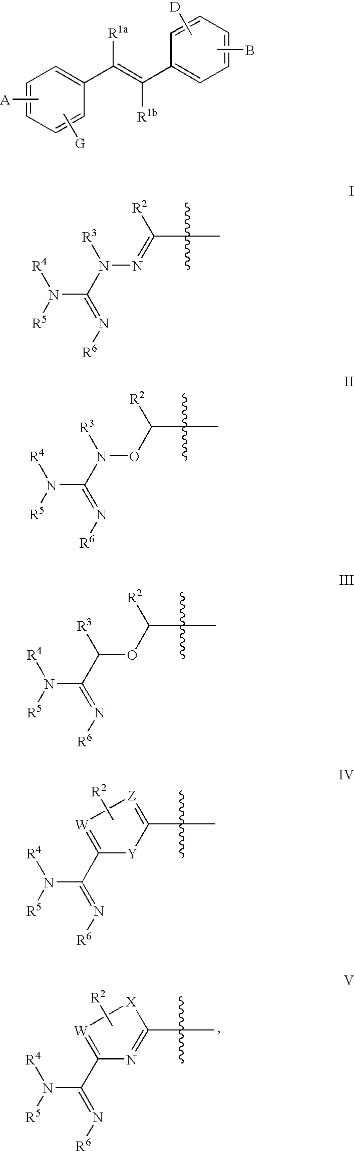
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R^{1a-1b} are independently hydrogen, alkyl, aralkyl, hydroxy, or alkoxy; R²⁻⁶ are independently hydrogen, alkyl, aryl, or aralkyl; W and Z are independently CH, C-alkyl, or N; and X and Y are independently NH, N-alkyl, O, or S. For example, R^{1a} is hydrogen, and R^{1b} is alkoxy.

In another aspect, the invention features a compound of the formula: 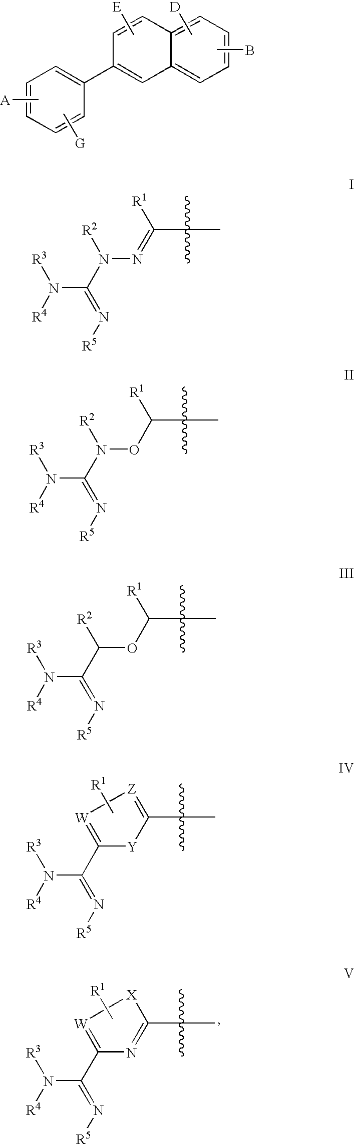
wherein A and B are independently selected from formulas I-V; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁵ are independently hydrogen, alkyl, aryl, or aralkyl; W and Z are independently CH, C-alkyl, or N; and X and Y are independently NH, N-alkyl, O, or S.

The invention further features a compound of the formula: 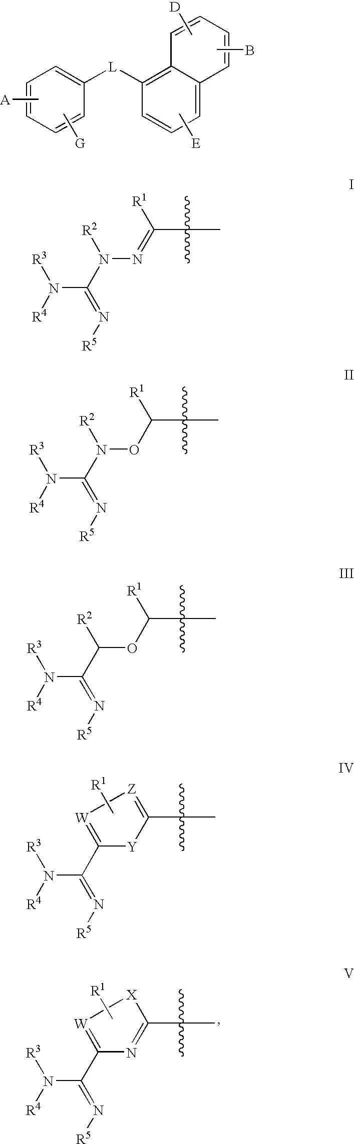
wherein A and B are independently selected from formulas I-V; L is O, S, CH₂, CHR⁶, or NR⁷; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁷ are independently hydrogen, alkyl, aryl, or aralkyl; W and Z are independently CH, C-alkyl, or N; and X and Y are independently NH, N-alkyl, O, or S.

In another aspect, the invention features a compound of the formula: 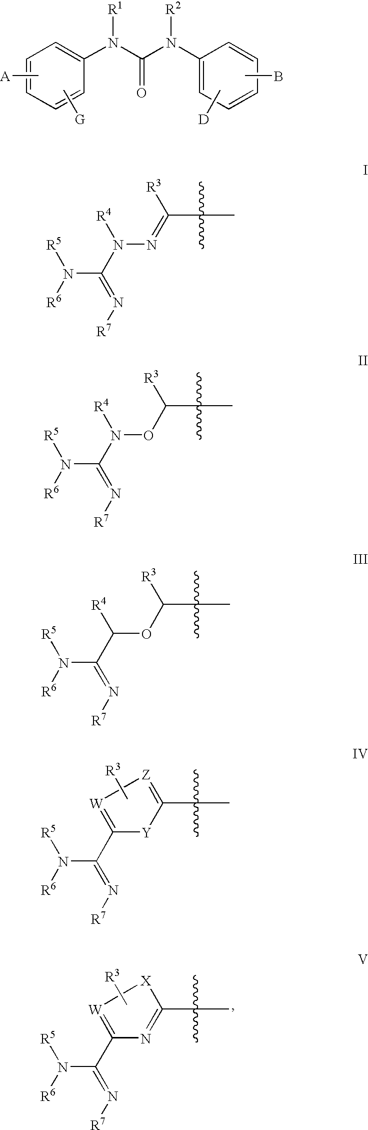
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁷ are independently hydrogen, alkyl, aryl, or aralkyl; W and Z are independently CH, C-alkyl, or N; and X and Y are independently NH, N-alkyl, O, or S.

Exemplary compounds of this formula include:

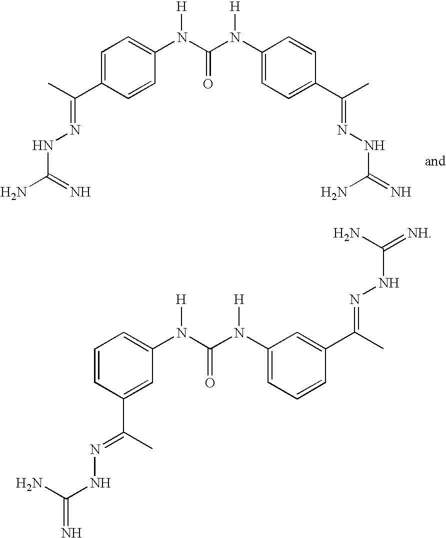

Additional compounds of the invention are of the formula: 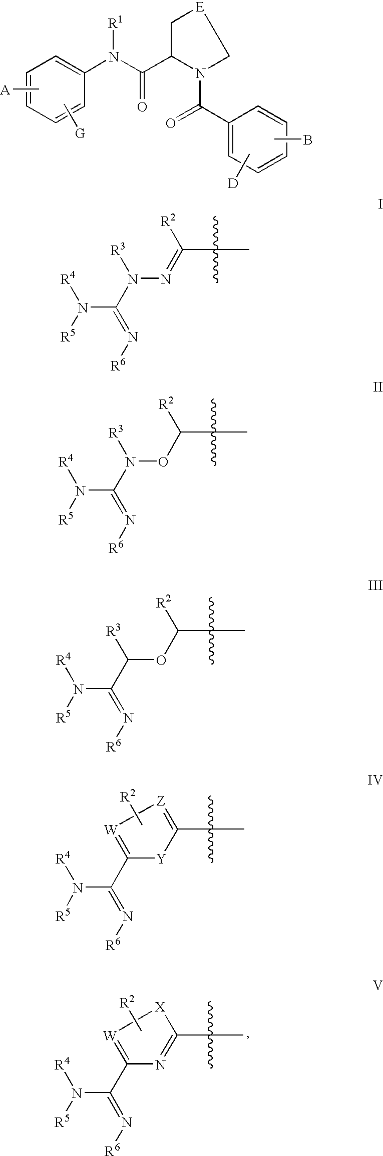
wherein A and B are independently selected from formulas I-V; E is (CH₂)ₙ, where n is 1-4, OCH₂, OCH₂CH₂, NR¹CH₂ or NR¹CH₂CH₂; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁶ are independently hydrogen, alkyl, aryl, or aralkyl; W and Z are independently CH, C-alkyl, or N; and X and Y are independently NH, N-alkyl, O, or S.

One example of compounds of this formula is

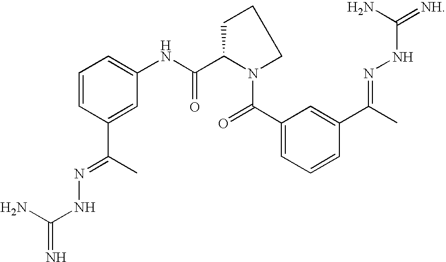

The invention also features compounds of the formula: 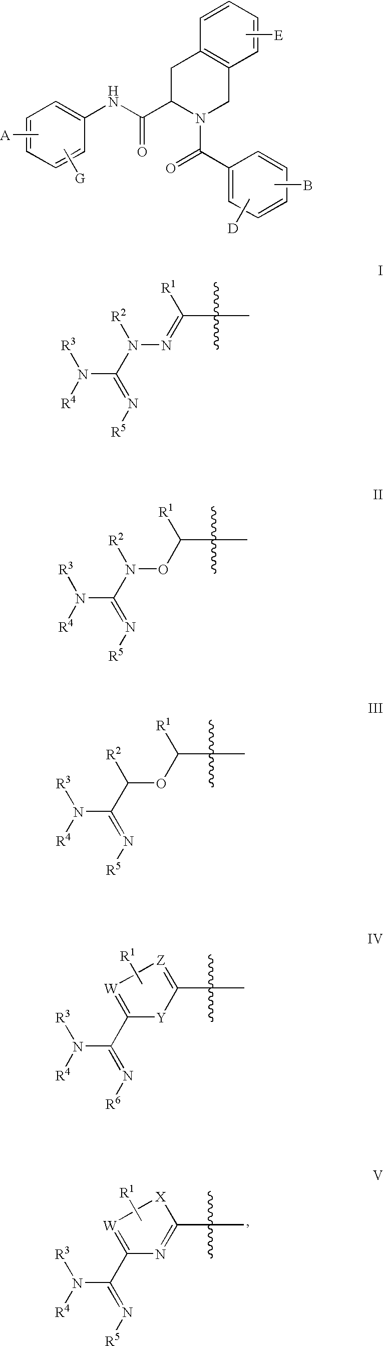
wherein A and B are independently selected from formulas I-V; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁶ are independently hydrogen, alkyl, aryl, or aralkyl; W and Z are independently CH, C-alkyl, or N; and X and Y are independently NH, N-alkyl, O, or S.

Exemplary compounds of this formula include:

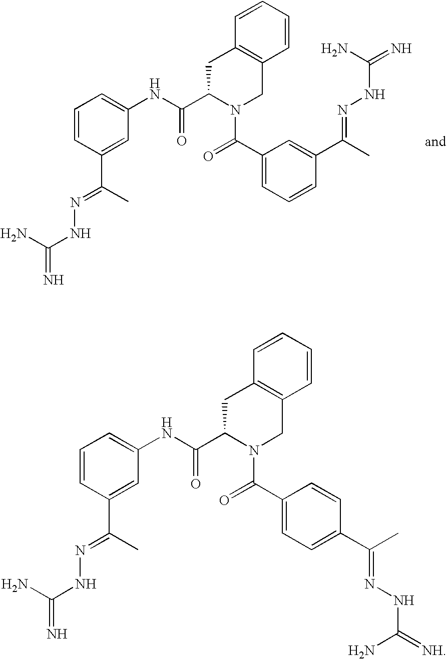

In various embodiments of the above aspects, A and B are formula I.

In another aspect, the invention features a compound of the formula: 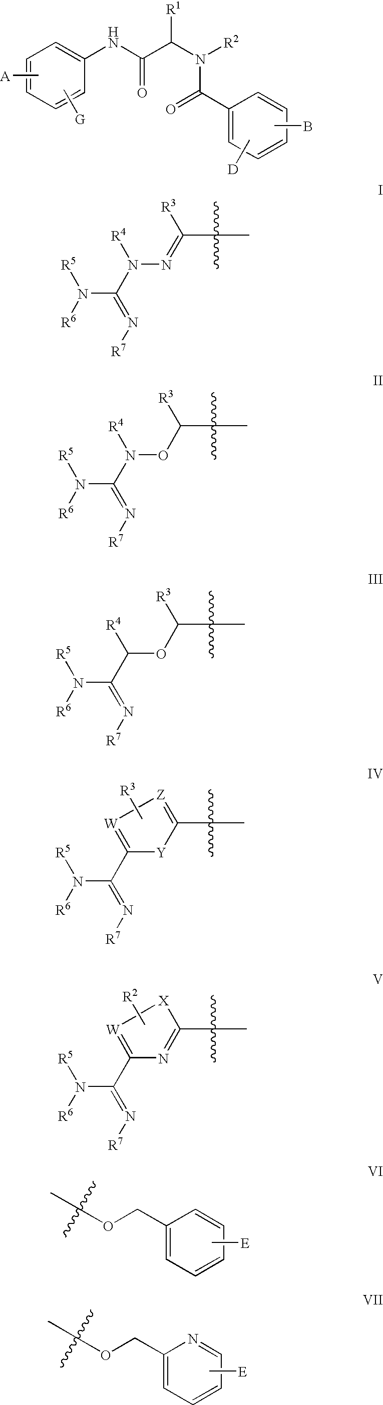 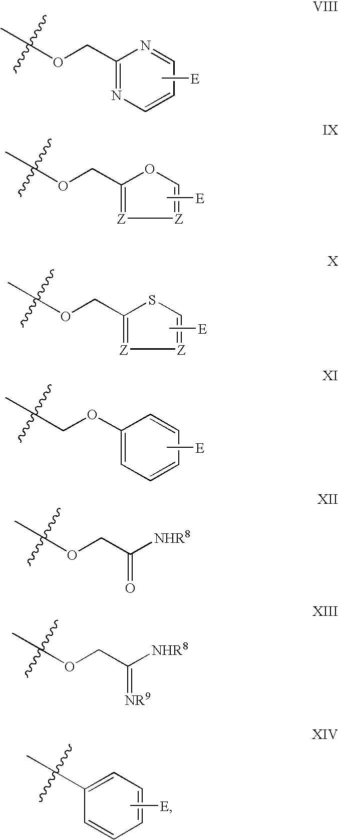
wherein A is selected from formulas I-V, and B is selected from hydrogen, halide, or formulas VI-XIV, or B is selected from formulas I-V, and A is selected from hydrogen, halide, or formulas VI-XIV; and wherein D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁹ are independently hydrogen, alkyl, aryl, or aralkyl; W and Z are independently CH, C-alkyl, or N; and X and Y are independently NH, N-alkyl, O, or S.

Exemplary compounds of this formula are

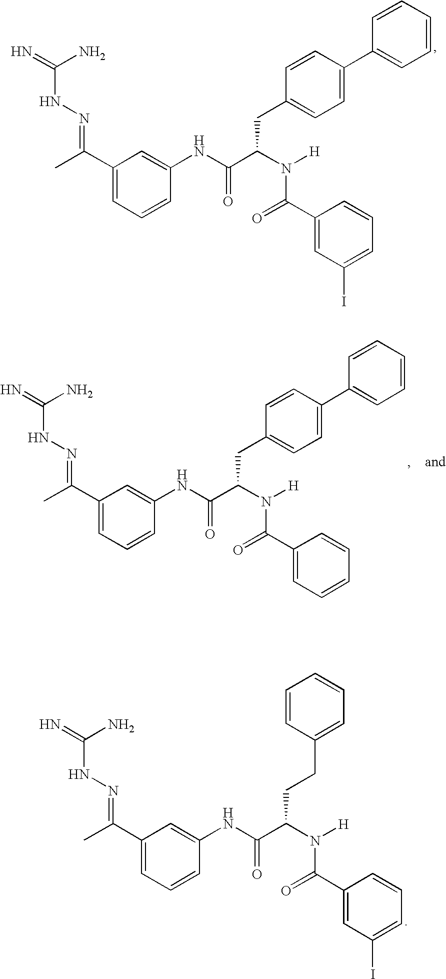

The invention further features compounds of the formula: 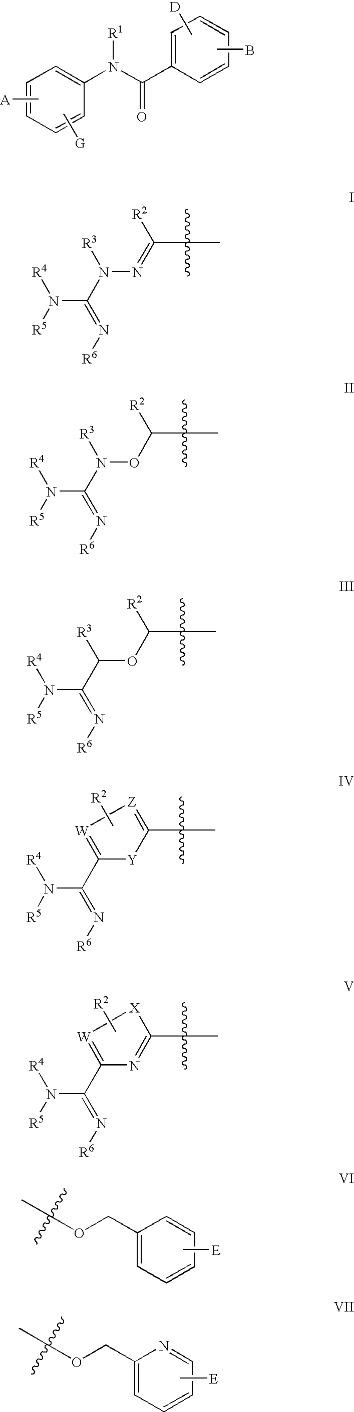 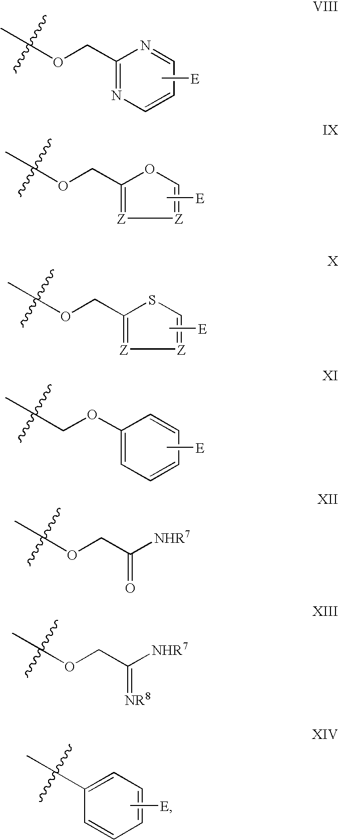
wherein A is selected from formulas I-V, and B is selected from hydrogen, halide, or formulas VI-XIV, or B is selected from formulas I-V, and A is selected from hydrogen, halide, or formulas VI-XIV; and wherein D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; and R¹⁻⁸ are independently hydrogen, alkyl, aryl, or aralkyl; W and Z are independently CH, C-alkyl, or N; and X and Y are independently NH, N-alkyl, O, or S.

Exemplary compounds of this formula include

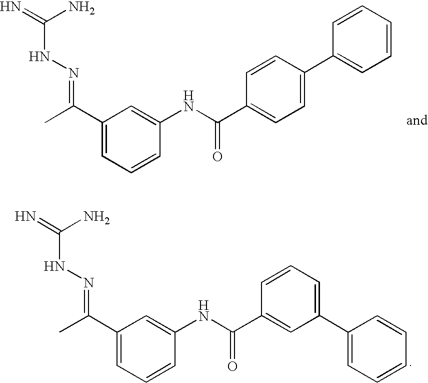

In yet another aspect, the invention features compounds of the formula: 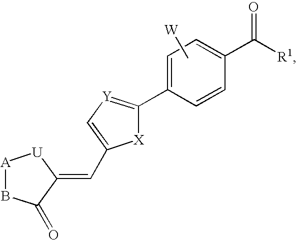
wherein R¹ is hydroxy, NHOR², NHNR³R⁴, or NR⁵OH, wherein R²⁻⁵ are independently hydrogen, lower alkyl, or aryl; W is hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is O, S, or NR⁶, wherein R⁶ is hydrogen or lower alkyl; Y is N, CH, or CR⁷, wherein R⁷ is hydrogen or lower alkyl; U is O, S, or NR⁸, wherein R⁸ is hydrogen, lower alkyl, or aryl; and A and B are independently selected from formulas I-VI, 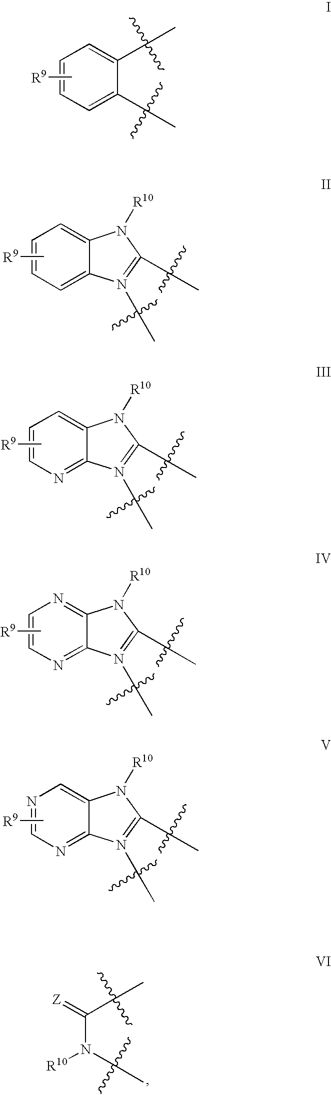
wherein R⁹ is hydrogen, alkyl, aralkyl, heteroalkyl, alkene, heteroalkene, alkyne, heteroalkyne, aryl, heteroaryl, alkoxy, halogen, amino, or alkylamino; and R¹⁰ is hydrogen, lower alkyl, or aryl; Z is O, S, or NR¹¹, wherein R¹¹ is hydrogen, lower alkyl, or aryl.

Exemplary compounds of this formula include:

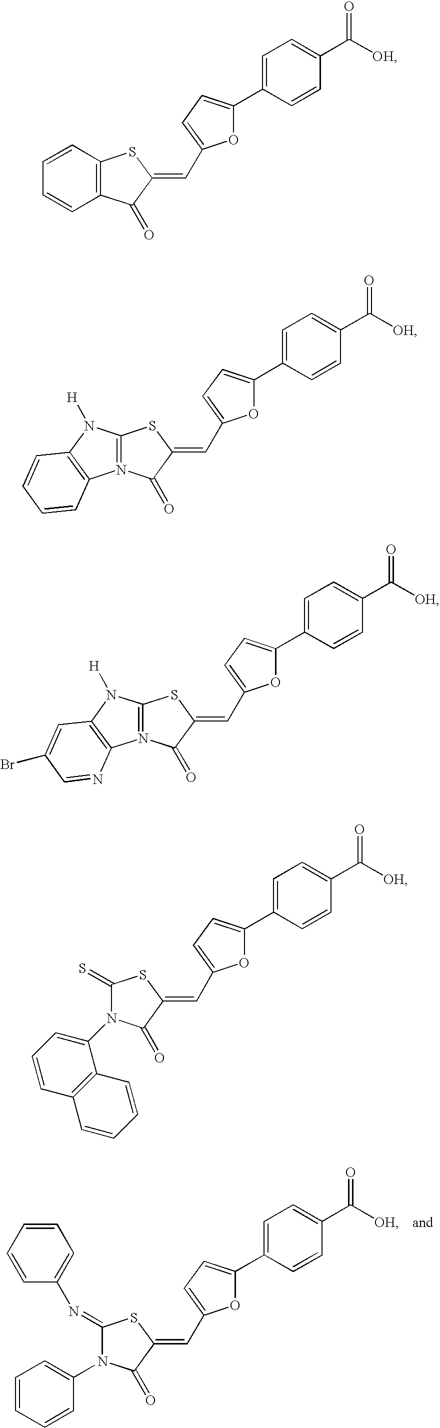

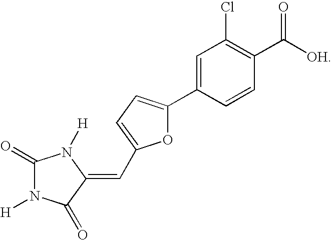

In another aspect, the invention features a compound of the formula: 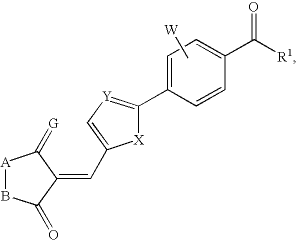
wherein R¹ is hydroxy, NHOR², NHNR³R⁴, or NR⁵OH, wherein R²⁻⁵ are independently hydrogen, lower alkyl, or aryl; W is hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is O, S, or NR⁶, wherein R⁶ is hydrogen or lower alkyl; Y is N, CH, or CR⁷, wherein R⁷ is hydrogen or lower alkyl; G is O, S, or NR⁸, wherein R⁸ is hydrogen, lower alkyl, or aryl; and A and B are independently selected from formulas I-IV, 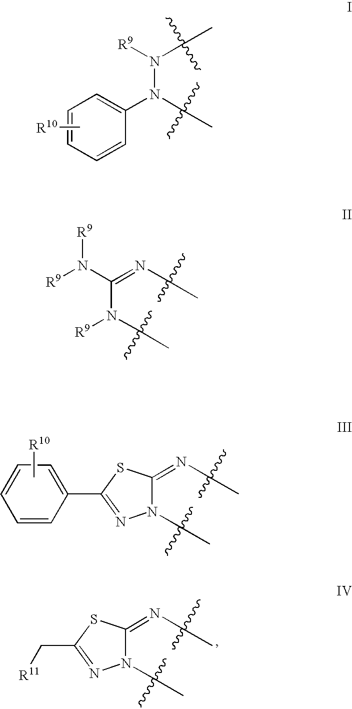
wherein R⁹ is hydrogen, lower alkyl, or aryl; R¹⁰ is hydrogen, alkyl, aralkyl, heteroalkyl, alkene, heteroalkene, alkyne, heteroalkyne, aryl, heteroaryl, alkoxy, halogen, amino, or alkylamino; and R¹¹ is hydrogen, lower alkyl, aryl or heteroaryl.

Exemplary compounds of this formula include:

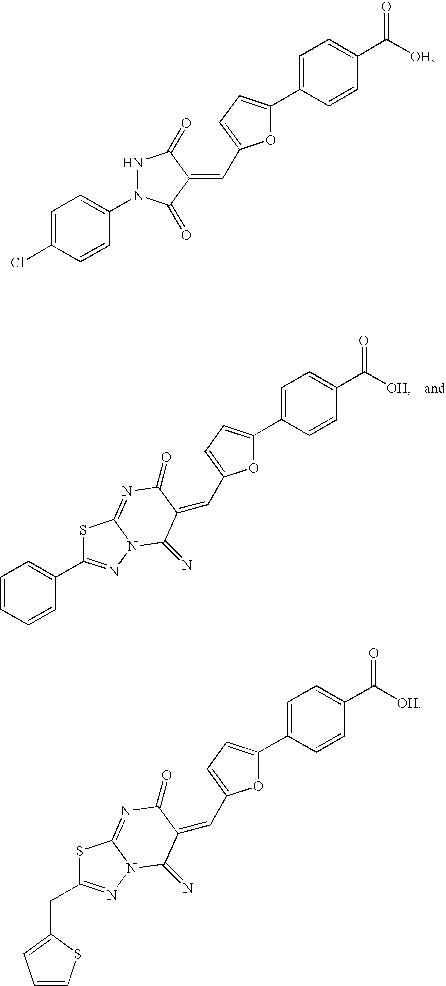

Additional compounds of the invention have the formula: 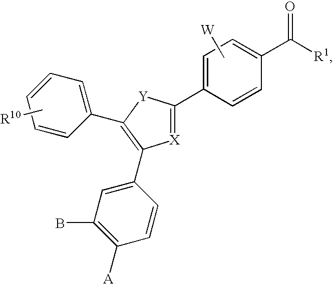
wherein R¹ is OH, NHOR², NHNR³R⁴, or NR⁵OH, wherein R²⁻⁵ are independently hydrogen, lower alkyl, or aryl; W and R¹⁰ are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is N, CH, or CR⁷, wherein R⁷ is hydrogen or lower alkyl; Y is O, S, or NR⁶, wherein R⁶ is hydrogen or lower alkyl; R⁸ is hydrogen, lower alkyl, or aryl; and B is hydrogen and A is aryl, heteroaryl, or 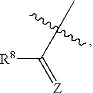
wherein Z is O, S, NR⁹, NNHR⁹, or NOR⁹, wherein R⁹ is hydrogen, lower alkyl, or aryl, or A and B together are

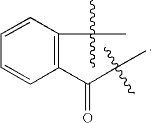

Exemplary compounds of this formula include:

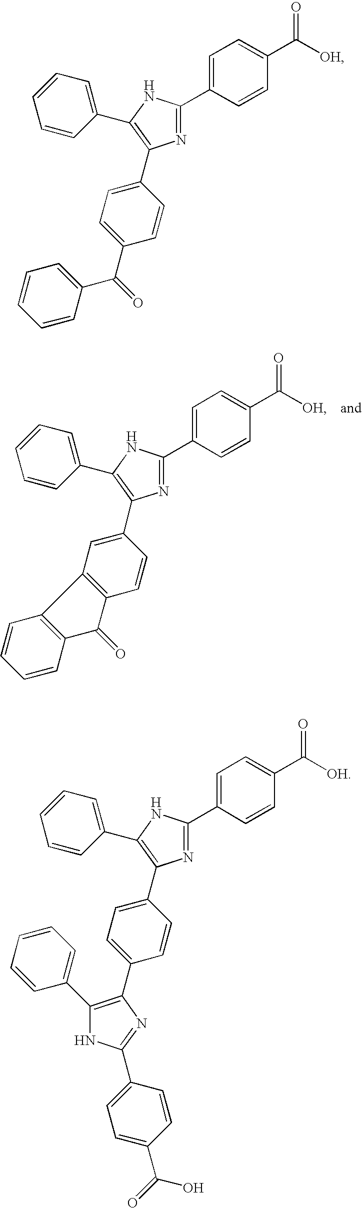

In still another aspect, the invention features a compound of the formula: 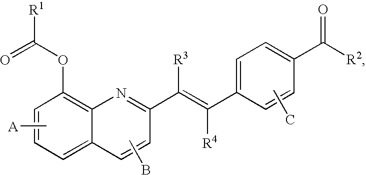
wherein R¹ is alkyl, aryl, or aralkyl; R² is OH, NHOR⁵, NHNR⁶R⁷, or NR⁸OH, wherein R⁵⁻⁸ are independently hydrogen, lower alkyl, or aryl; R³ and R⁴ are hydrogen or alkyl; and A, B, and C are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio.

An exemplary compound of the invention has the formula:

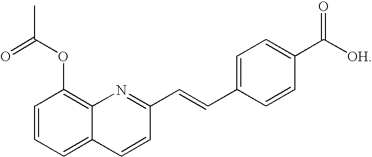

The invention also features a compound of the formula: 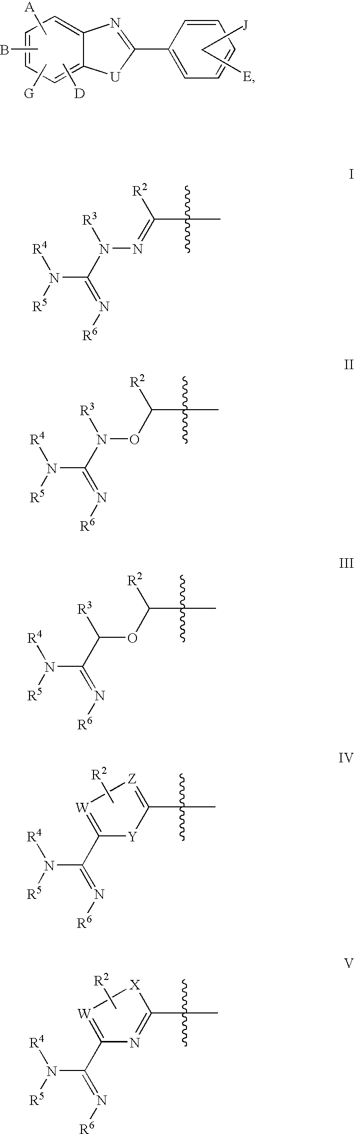
wherein U is O, S, or NR⁷; A and E are independently selected from formulas I-V; B, D, G, and J are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R²⁻⁷ are independently hydrogen, alkyl, aryl, or aralkyl; W and Z are independently CH, C-alkyl, or N; and X and Y are independently NH, N-alkyl, O, or S. For example, a compound of the formula: 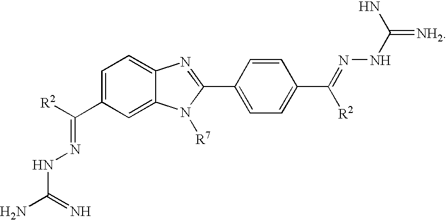
   Alternatively, the guanyl hydrazone is in the meta position on the phenyl ring.

Exemplary compounds of these formulas include:

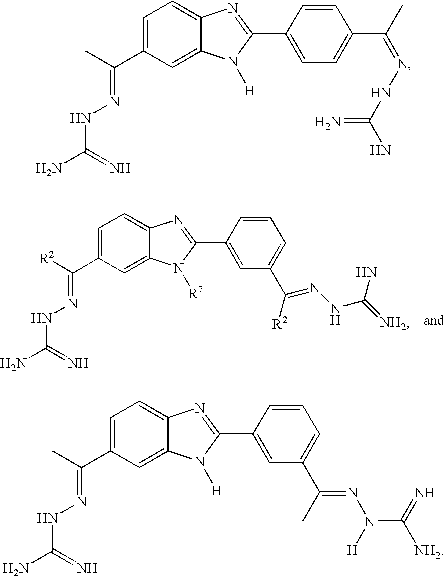

In another aspect the invention features a pharmaceutical composition including a pharmaceutically acceptable carrier and any one or more of the compounds of invention. In one embodiment, a pharmaceutical composition includes a composition of the invention as the only active ingredient. The invention also features any of the compounds of the invention in substantially pure form, e.g., as at least 10, 20, 30, 40, 50, 60, 70, 75, 80, 85, 90, 95, or even 99% of a composition by weight. A substantially pure compound of the invention may be used in any of the methods described herein or in a pharmaceutical composition as described herein.

In yet another aspect, the invention features a method of killing or inhibiting the growth of bacteria that includes contacting bacteria or a site susceptible to bacterial growth, e.g., an in-dwelling device in a patient, with a pharmaceutical composition as described herein. In various embodiments, the contacting is administering the pharmaceutical composition to a mammal, e.g., a human. The pharmaceutical composition is, for example, administered to the skin, hair, oral cavity, a mucous membrane, a wound, a bruise, a tooth, or an eye. The site susceptible bacterial growth may be, for example, an in-dwelling device in a patient, a medical device, a food, beverage, cosmetic deodorant, contact lens product, food ingredient, enzyme compositions, a hard surface, or laundry. In various embodiments, the compound in the pharmaceutical composition inhibits a bacterial RNase P enzyme.

In desirable embodiments of any of the above aspects, the compound inhibits RNase P activity in vitro or in vivo, e.g., by at least 10%, 20%, 30%, 40%, 50%, 60%, 70%, 80%, 90%, 95%, 98% or 100%. In various embodiments, the compound specifically inhibits one RNase P holoenzyme or inhibits multiple RNase P holoenzymes from different bacterial genera, species, or strain. In one embodiment, the compound inhibits the activity of RNase P from one bacterial species by at least 2, 5, 10, 20, 50, 100, 500, or 1000 fold more than it inhibits the activity of RNase P from another genus, species, or strain of bacteria.

In an embodiment of any of the above aspects, the step of contacting bacteria or a site susceptible to bacterial growth with the compound includes using one or more compounds of the invention as an antibacterial ingredient wherever such an ingredient is needed. For example, a compound of the invention can be used for the preservation of food, beverages, cosmetics, deodorants, contact lens products, food ingredients or enzyme compositions. Alternatively, a compound of the invention can be used as a disinfectant for use, e.g., on human or animal skin, hair, oral cavity, mucous membranes, wounds, bruises, or in the eye. In other embodiments, the compound is used for killing bacterial cells in laundry; or is incorporated into cleaning compositions or disinfectants for hard surface cleaning or for water treatment.

Accordingly, in further aspects, the invention provides a method of inhibiting bacteria present in laundry by treating the laundry with a soaking, washing, or rinsing liquor that includes a compound of the invention; a method of inhibiting bacterial growth on a hard surface by contacting the surface with a compound of the invention; a method of inhibiting bacterial growth present in an industrial water line by contacting the water line with a compound of the invention; and a method of killing bacterial cells on human or animal skin, mucous membranes, teeth, wounds, bruises or in the eye or inhibiting the growth thereof by administering a compound of the invention to the relevant site on or in the animal.

In a further embodiment of any of the above aspects, the step of contacting bacteria or a site susceptible to bacterial growth with the compound includes contacting an in-dwelling device with the compound prior to, concurrent with, or following the administration of the in-dwelling device to a patient. In-dwelling devices include, but are not limited to, surgical implants, prosthetic devices, and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters.

In another embodiment of any of the above aspects, the method is used to treat, stabilize or prevent a bacterial infection in a mammal. In this method, the step of contacting bacteria or a site susceptible to bacterial infection (e.g., a site in or on the body of mammal) with the compound includes administering to the mammal the compound in an amount sufficient to treat, stabilize, or prevent the bacterial infection in the mammal.

In various embodiments of the invention, the mammal is a human, an animal of veterinary interest (e.g., cow, horse, dog, pig, sheep, or cat), or any other mammalian species.

In the desirable embodiments, the bacterial RNase P to be targeted by a compound of the invention is taken from a bacterium selected from the group consisting of Chlamydophila pneumoniae, C. psittaci, C. abortus, Chlamydia trachomatis, Simkania negevensis, Parachlamydia acanthamoebae, Pseudomonas aeruginosa, P. alcaligenes, P. chlororaphis, P. fluorescens, P. luteola, P. mallei, P. mendocina, P. monteilii, P. oryzihabitans, P. pertocinogena, P. pseudalcaligenes, P. putida, P. stutzeri, Burkholderia cepacia, B. pseudomallei, Aeromonas hydrophilia, Escherichia coli, Citrobacter freundii, Salmonella typhimurium, S. typhi, S. paratyphi, S. enteritidis, Shigella dysenteriae, S. flexneri, S. sonnei, Enterobacter cloacae, E. aerogenes, Klebsiella pneumoniae, K. oxytoca, Serratia marcescens, Francisella tularensis, Morganella morganii, Proteus mirabilis, Proteus vulgaris, Providencia alcalifaciens, P. rettgeri, P. stuartii, Acinetobacter calcoaceticus, A. haemolyticus, Yersinia enterocolitica, Y. pestis, Y. pseudotuberculosis, Y. intermedia, Bordetella pertussis, B. parapertussis, B. bronchiseptica, Haemophilus influenzae, H. parainfluenzae, H. haemolyticus, H. parahaemolyticus, H. ducreyi, Pasteurella multocida, P. haemolytica, Branhamella catarrhalis, Brusella spp. (e.g., B. abortus), Helicobacter pylori, Campylobacter fetus, C. jejuni, C. coli, Borrelia burgdorferi, V. cholerae, V. parahaemolyticus, Legionella pneumophila, Listeria monocytogenes, Neisseria gonorrhea, N. meningitidis, Kingella dentrificans, K. kingae, K. oralis, Moraxella catarrhalis, M. atlantae, M. lacunata, M. nonliquefaciens, M. osloensis, M. phenylpyruvica, Gardnerella vaginalis, Bacillus anthracis, Bacteroides fragilis, Bacteroides distasonis, Bacteroides 3452A homology group, Bacteroides vulgatus, B. ovalus, B. thetaiotaomicron, B. uniformis, B. eggerthii, B. splanchnicus, Coxiella burnetti, Clostridium difficile, C. diphtheriae, C. ulcerans, C. accolens, C. afermentans, C. amycolatum, C. argentorense, C. auris, C. bovis, C. confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Mycobacterium tuberculosis, M. avium, M. intracellulare, M. leprae, Streptococcus pneumoniae, S. agalactiae, S. pyogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Staphylococcus aureus, S. epidermidis, S. saprophyticus, S. intermedius, S. hyicus, S. haemolyticus, S. hominis, S. saccharolyticus, and Treponema pallidum (e.g., subspecies pertenue). Accordingly, the invention discloses a method of treating infections by the bacteria above, among others.

In another aspect, the invention features a pharmaceutical composition that includes a compound described herein in any pharmaceutically acceptable form, including isomers such as E/Z isomers, diastereomers, and enantiomers, salts, solvates, and polymorphs thereof. In various embodiments, the composition includes a compound of the invention along with a pharmaceutically acceptable carrier or diluent.

By “alkyl” is meant a branched or unbranched saturated hydrocarbon group, desirably having from 1 to 20 or 1 to 50 carbon atoms. An alkyl may optionally include monocyclic, bicyclic, or tricyclic rings, in which each ring desirably has three to six members. The alkyl group may be substituted or unsubstituted. Exemplary substituents include alkoxy, aryloxy, sulflhydryl, alkylthio, arylthio, halogen, hydroxy, fluoroalkyl, perfluoralkyl, amino, alkylamino, disubstituted amino, quaternary amino, hydroxyalkyl, aryl, and carboxyl groups.

In various embodiments of the invention the alkyl group is of 1 to 5, 1 to 7, 1 to 10, 1 to 15, 1 to 20, 1 to 50, 5 to 10, 5 to 15, 5 to 50, 10 to 15, 10 to 35, or 10 to 50 carbon atoms. Exemplary alkyl groups include methyl; ethyl; n-propyl; isopropyl; n-butyl; iso-butyl; sec-butyl; tert-butyl; pentyl; cyclopropyl; cyclobutyl; cyclopentyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; hexyl; heptyl; cyclohexyl; cycloheptyl; and cyclooctyl.

By “alkene” is meant a branched or unbranched hydrocarbon group containing one or more double bonds, desirably having from 2 to 20 or 2 to 50 carbon atoms. An alkene may optionally include monocyclic, bicyclic, or tricyclic rings, in which each ring desirably has five or six members. The alkene group may be substituted or unsubstituted. Exemplary substituents include alkoxy, aryloxy, sulfhydryl, alkylthio, arylthio, halogen, hydroxy, fluoroalkyl, perfluoralkyl, amino, alkylamino, disubstituted amino, quaternary amino, hydroxyalkyl, and carboxyl groups.

In various embodiments of the invention the alkene group is of 2 to 5, 2 to 7, 2 to 10, 2 to 15, 2 to 20, 2 to 50, 5 to 10, 5 to 15, 5 to 50, 10 to 15, 10 to 35, or 10 to 50 carbon atoms. Exemplary alkenyl groups include vinyl; allyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “alkyne” is meant a branched or unbranched hydrocarbon group containing one or more triple bonds, desirably having from 2 to 20 or 2 to 50 carbon atoms. An alkyne may optionally include monocyclic, bicyclic, or tricyclic rings, in which each ring desirably has five or six members. The alkyne group may be substituted or unsubstituted. Exemplary substituents include alkoxy, aryloxy, sulfhydryl, alkylthio, arylthio, halogen, hydroxy, fluoroalkyl, perfluoralkyl, amino, alkylamino, disubstituted amino, quaternary amino, hydroxyalkyl, and carboxyl groups.

In various embodiments of the invention the alkyne group is of 2 to 5, 2 to 7, 2 to 10, 2 to 15, 2 to 20, 2 to 50, 5 to 10, 5 to 15, 5 to 50, 10 to 15, 10 to 35, or 10 to 50 carbon atoms. Exemplary alkynyl groups include ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “heteroalkyl” is meant a branched or unbranched group, having from 1 to 50 atoms selected from the group consisting of carbon, nitrogen, oxygen, sulfur, or phosphorous. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halogen, hydroxy, fluoroalkyl, perfluoralkyl, amino, alkylamino, disubstituted amino, quaternary amino, hydroxyalkyl, and carboxyl groups.

By “heteroalkene” is meant a branched or unbranched group containing one or more double bonds, desirably having from 2 to 20 or 2 to 50 atoms selected from the group consisting of carbon, nitrogen, oxygen, sulfur, and phosphorous. A heteroalkene may optionally include monocyclic, bicyclic, or tricyclic rings, in which each ring desirably has five or six members. The heteroalkene group may be substituted or unsubstituted. Exemplary substituents include alkoxy, aryloxy, sulfhydryl, alkylthio, arylthio, halogen, hydroxy, fluoroalkyl, perfluoralkyl, amino, alkylamino, disubstituted amino, quaternary amino, hydroxyalkyl, and carboxyl groups.

By “heteroalkyne” is meant a branched or unbranched group containing one or more triple bonds, desirably having from 2 to 50 atoms selected from the group consisting of carbon, nitrogen, oxygen, sulfur, and phosphorous. A heteroalkyne may optionally include monocyclic, bicyclic, or tricyclic rings, in which each ring desirably has five or six members. The alkyne group may be substituted or unsubstituted. Exemplary substituents include alkoxy, aryloxy, sulfhydryl, alkylthio, arylthio, halogen, hydroxy, fluoroalkyl, perfluoralkyl, amino, alkylamino, disubstituted amino, quaternary amino, hydroxyalkyl, and carboxyl groups.

By “aryl” is meant an aromatic group having a ring system comprised of carbon atoms with conjugated π electrons (e.g., phenyl). The ring of the aryl group is desirably 6 to 18 atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulfhydryl, alkylthio, arylthio, halogen, fluoroalkyl, carboxyl, amino, alkylamino, monosubstituted amino, disubstituted amino, and quaternary amino groups. Exemplary aryl groups include phenyl, naphthyl, biphenyl, indenyl, pentalenyl, azulenyl, anthranyl, and substituted variants thereof.

By “heteroaryl” is meant an aromatic group having a ring system with conjugated π electrons (e.g., imidazole). The ring of the heteroaryl group is desirably 5 to 18 atoms selected from the group consisting of carbon, nitrogen, oxygen, sulfur, and phosphorous. Heteroaryl groups may optionally include monocyclic, bicyclic, or tricyclic rings, in which each ring desirably has five or six members. The heteroaryl group may be substituted or unsubstituted. Exemplary substituents include alkyl, hydroxy, alkoxy, aryloxy, sulfhydryl, alkylthio, arylthio, halogen, fluoroalkyl, carboxyl, amino, alkylamino, monosubstituted amino, disubstituted amino, and quaternary amino. Exemplary heterocyclic groups include pyranyl, pyrrolyl, pyrazolyl, pyridyl, quinolyl, isoquinolyl, indolyl, isoindolyl, indazolyl, purinyl, phthalazinyl, triazolyl, imidazolyl, pyrazinyl, pyrimidinyl, pyridazinyl, indolizinyl, and substituted variants thereof.

By “fluoroalkyl” is meant an alkyl group that is substituted with one or more fluorine atoms.

By “perfluoroalkyl” is meant an alkyl group consisting of only carbon and fluorine atoms.

By “hydroxyalkyl” is meant a chemical moiety with the formula —(R)—OH, wherein R is an alkyl group.

By “alkoxy” is meant a chemical substituent of the formula —OR, wherein R is an alkyl group.

By “aryloxy” is meant a chemical substituent of the formula —OR, wherein R is an aryl group.

By “alkylthio” is meant a chemical substituent of the formula —SR, wherein R is an alkyl group.

By “arylthio” is meant a chemical substituent of the formula —SR, wherein R is an aryl group.

By “alkylamino” is meant a chemical substituent of the formula —NR′R″, wherein at least one of R′ and R″ is an alkyl group and the other group is hydrogen or alkyl.

By “aralkyl” is meant a chemical substituent of the formula —R′—R″, wherein R′ is alkyl and R″ is aryl.

By “halogen” is meant fluorine, chlorine, bromine, or iodine.

By “quaternary amino” is meant a chemical substituent of the formula —(R)—N(R′)(R″)(R′″)⁺, wherein R, R′, R″, and R′″ are each independently an alkyl, alkene, alkyne, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

By “inhibiting bacterial growth” is meant preventing, reducing the rate or extent of, or stabilizing bacterial replication. By “stabilizing bacterial replication” is meant maintaining a bacterial population at an approximately constant level.

By inhibiting “RNase P activity” is meant decreasing the amount of an activity of an RNase P enzyme. For example, the amount of 5′ terminal leader sequences that are cleaved from precursor tRNA's may be decreased. In various embodiments, the amount of an RNase P substrate (e.g., ptRNA^{Gln}) that is hydrolyzed in vitro or in vivo is decreased by at least 10%, 20%, 30%, 40%, 50%, 60%, 70%, 80%, 90%, 95%, or 100% relative to a corresponding control without an RNase P inhibitor. In other embodiments, the percentage of fluorescence in the presence of a candidate compound in comparison to the absence of the candidate compound is less than 90%, 80%, 70%, 60%, 50%, 40%, 30%, 20%, 10%, 5% or 2%, as calculated using equation 1, with solutions prepared as described in herein. In other embodiments, the level of RNase P activity is at least 2, 5, 10, or 20 fold lower in the presence of a candidate inhibitor than in the absence of the candidate inhibitor. In another embodiment, a compound decreases RNase P activity by inhibiting assembly of the RNase P holoenzyme. In still other embodiments, a compound decreases RNase P activity by inhibiting the binding of RNase P (RNA subunit, or protein subunit, or holoenzyme) to another molecule (e.g., a substrate); or the enzymatic activity of an RNase P holoenzyme, as measured using standard assays such as these described herein or any other standard assay (see, for example, Ausubel et al., Current Protocols in Molecular Biology, Wiley: New York, 2000).

By “treating” is meant administering a pharmaceutical composition for prophylactic and/or therapeutic purposes. To “prevent disease” refers to prophylactic treatment of a subject who is not yet infected, but who is susceptible to, or otherwise at risk of, a particular infection. To “treat disease” or use for “therapeutic treatment” refers to administering treatment to a subject already suffering from an infection to improve the subject's condition.

By “effective amount” is meant an amount of a compound sufficient to kill bacteria or inhibit bacterial growth. This amount may vary from compound to compound and may depend on the route of administration.

By “bacterial infection” is meant the invasion of a host animal, e.g., a mammal, by pathogenic bacteria. For example, the infection may include the excessive growth of bacteria that are normally present in or on the body of a mammal or growth of bacteria that are not normally present in or on the mammal. More generally, a bacterial infection can be any situation in which the presence of a bacterial population(s) is damaging to a host mammal. Thus, a mammal is “suffering” from a bacterial infection when an excessive amount of a bacterial population is present in or on the mammal's body, or when the presence of a bacterial population(s) is damaging the cells or other tissue of the mammal. In one embodiment, the number of a particular genus or species of bacteria is at least 2, 4, 6, or 8 times the number normally found in the mammal. The bacterial infection may be due to gram positive and/or gram negative bacteria or any other class of bacteria.

By “administration” or “administering” is meant a method of giving one or more unit doses of an antibacterial pharmaceutical composition to an animal, e.g., a mammal (e.g., topical, oral, intravenous, intraperitoneal, or intramuscular administration). The method of administration may vary depending on various factors, e.g., the components of the pharmaceutical composition, site of the potential or actual bacterial infection, bacteria involved, and severity of the actual bacterial infection.

By “substantially pure” compound is meant a composition including at least 10% by weight of the compound.

The compounds of the invention that inhibit RNase P activity have a variety of advantages. For example, the inhibitors may provide a selective antibacterial treatment that reduces the adverse side effects associated with killing nonpathogenic bacteria. Use of such selective inhibitors also reduces the risk of producing a wide range of resistant bacterial strains.

Other features and advantages of the invention will be apparent from the following detailed description and from the claims.

### DETAILED DESCRIPTION OF THE INVENTION

We have identified compounds that inhibit RNase P activity and that are useful for killing bacteria or inhibiting bacterial growth, e.g., to treat or prevent infection. The compounds of the invention include guanylhydrazones (e.g., mono or bis) and benzoic acid compounds.

Exemplary bisguanylhydrazone inhibitors of RNase P activity of the invention have the following formula: 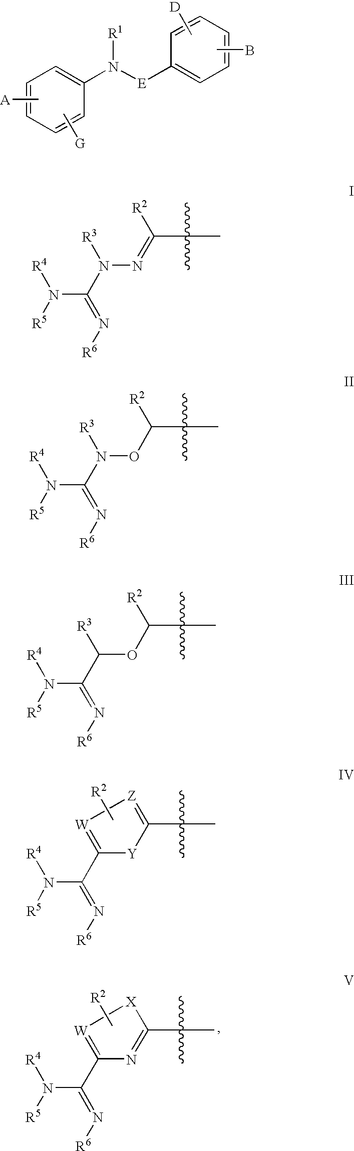
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; E is E is C═O, C═S, C═CR⁸R⁹, or C═NR⁷; R¹⁻⁹ are independently hydrogen, alkyl, aryl, or aralkyl; W and Z are independently CH, C-alkyl, or N; and X and Y are independently NH, N-alkyl, O, or S.

In this structure, an amide group is typically used to link the two guanylhydrazones. This linker may, however, be replaced by a variety of moieties, for example, to improve bioavailability, degradation characteristics, activity, ease of synthesis, or other factors. Exemplary alternative linkers include —NRC(NR)—, —NRC(S)—, —NRC(H₂)—, NRC(alkyl)₂-, substituted or unsubstituted ethylene, substituted or unsubstituted ethyl, or urea.

Alternative linkers are employed in the following formulas: 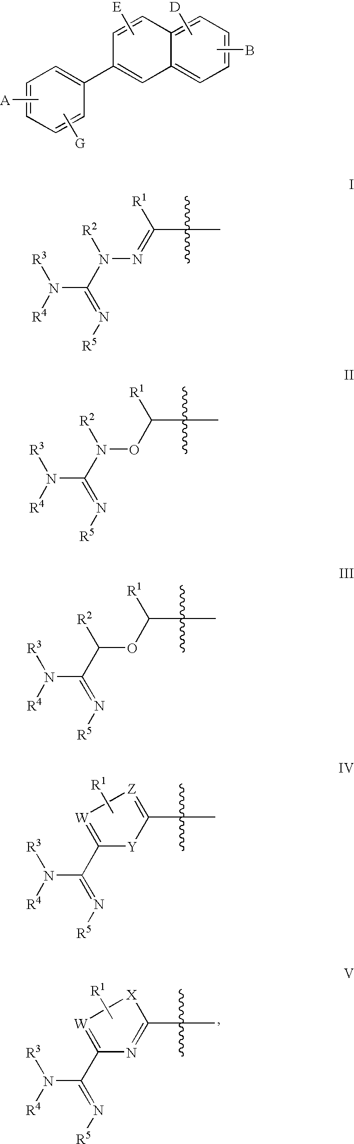
wherein A and B are independently selected from formulas I-V; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁵ are independently hydrogen, alkyl, aryl, or aralkyl; W and Z are independently CH, C-alkyl, or N; and X and Y are independently NH, N-alkyl, O, or S; 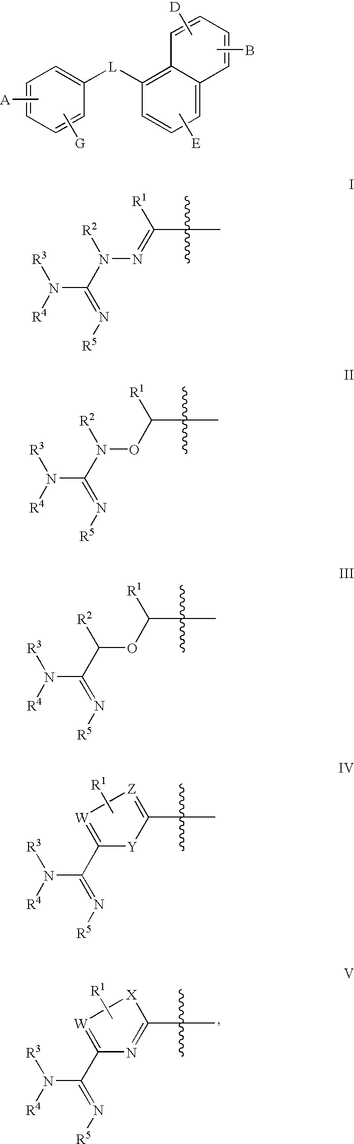
   wherein A and B are independently selected from formulas I-V; L is O, S, CH₂, CHR⁶, or NR⁷; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁷ are independently hydrogen, alkyl, aryl, or aralkyl; W and Z are independently CH, C-alkyl, or N; and X and Y are independently NH, N-alkyl, O, or S; 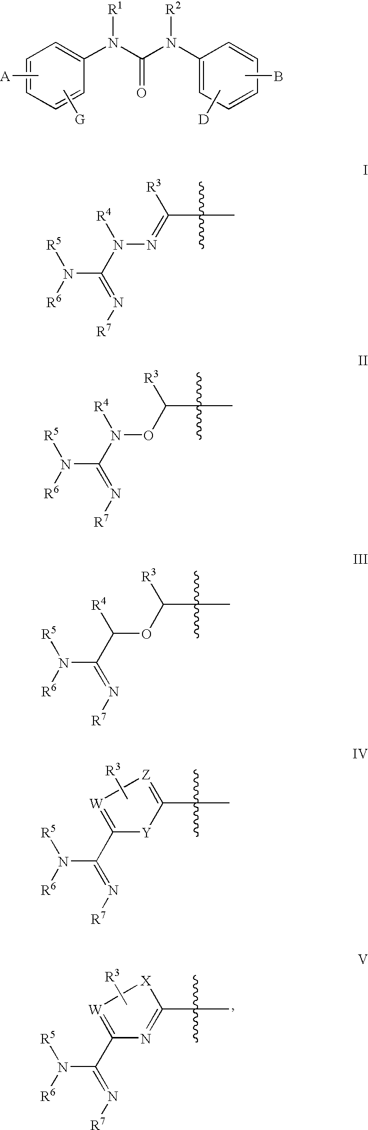
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁷ are independently hydrogen, alkyl, aryl, or aralkyl; W and Z are independently CH, C-alkyl, or N; and X and Y are independently NH, N-alkyl, O, or S; 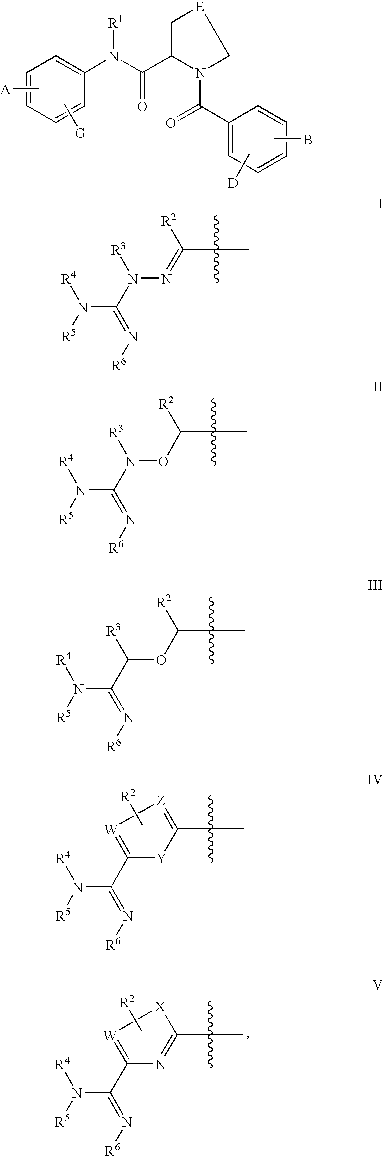
wherein A and B are independently selected from formulas I-V; E is (CH₂)ₙ, where n is 1-4, OCH₂, OCH₂CH₂, NR¹CH₂ or NR¹CH₂CH₂; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁶ are independently hydrogen, alkyl, aryl, or aralkyl; W and Z are independently CH, C-alkyl, or N; and X and Y are independently NH, 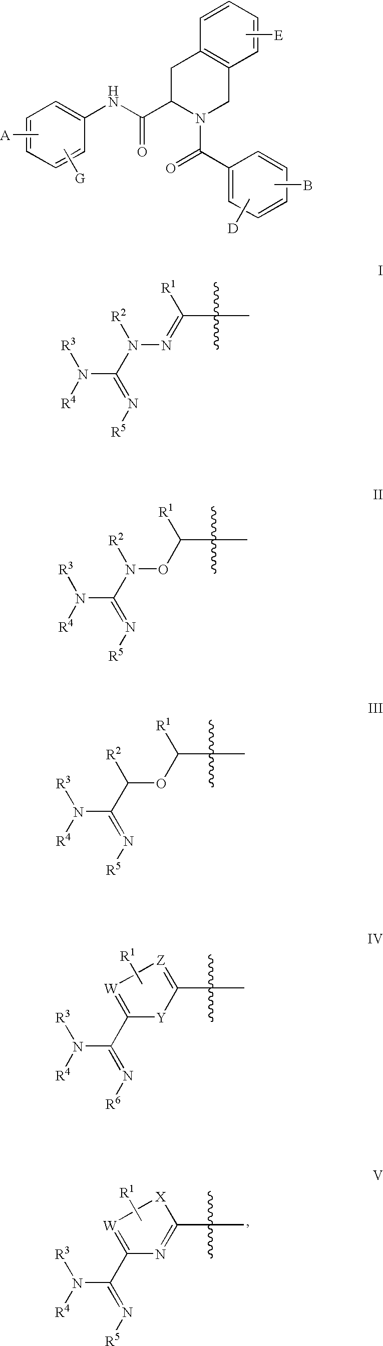
wherein A and B are independently selected from formulas I-V; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁶ are independently hydrogen, alkyl, aryl, aralkyl; W and Z are independently CH, C-alkyl, or N; and X and Y are independently NH, N-alkyl, O, or S; 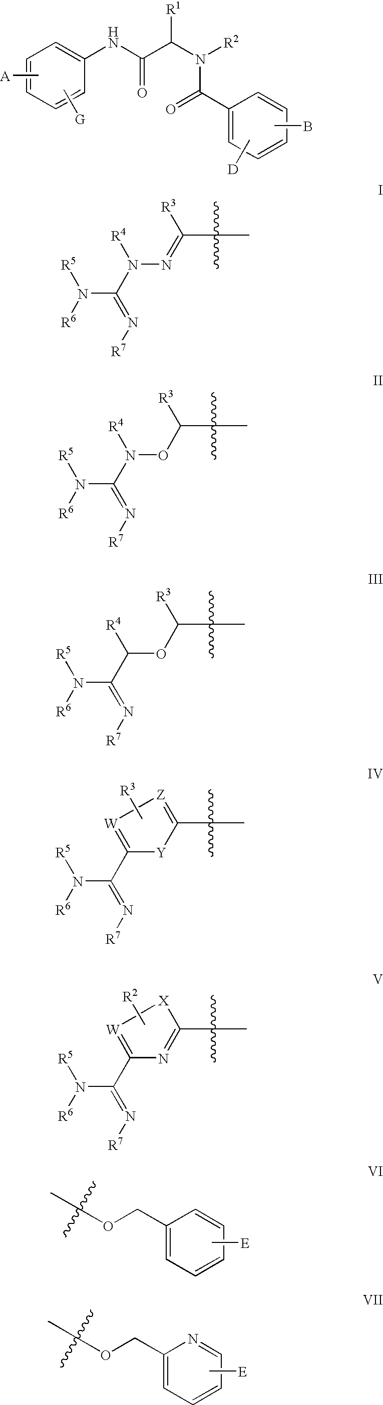 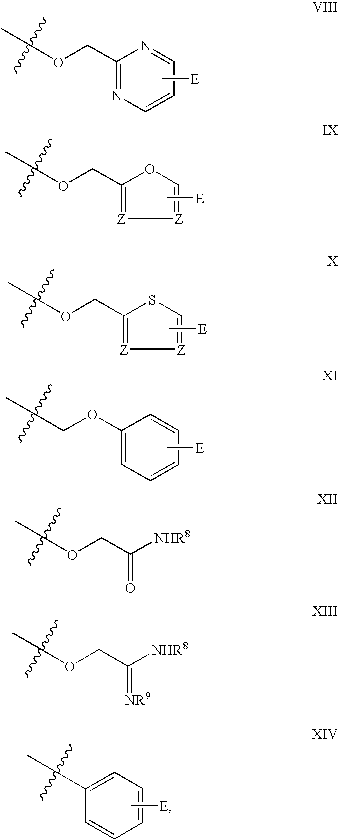
wherein A is selected from formulas I-V, and B is selected from hydrogen, halide, or formulas VI-XIV, or B is selected from formulas I-V, and A is selected from hydrogen, halide, or formulas VI-XIV; and wherein D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁹ are independently hydrogen, alkyl, aryl, or aralkyl; W and Z are independently CH, C-alkyl, or N; and X and Y are independently NH, N-alkyl, O, or S; and 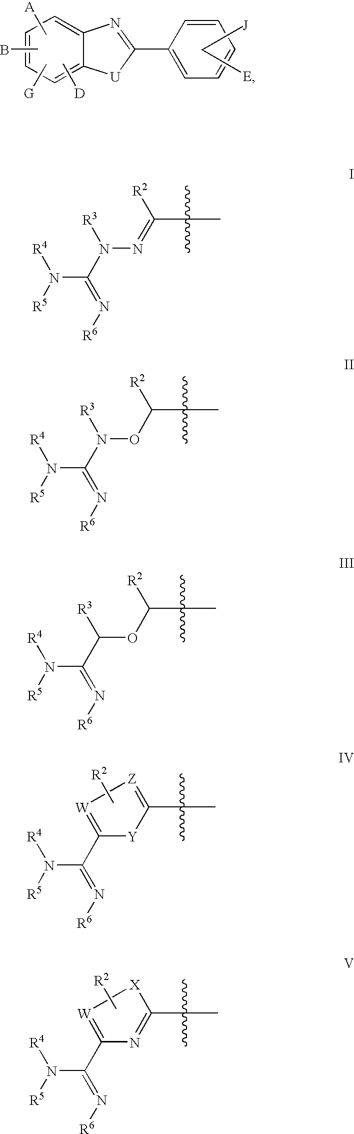
wherein U is O, S, or NR⁷; A and E are independently selected from formulas I-V; B, D, G, and J are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R²⁻⁷ are independently hydrogen, alkyl, aryl, or aralkyl; W and Z are independently CH, C-alkyl, or N; and X and Y are independently NH, N-alkyl, O, or S.

Exemplary benzoic acid inhibitors of RNase P activity of the invention have the following formulas: 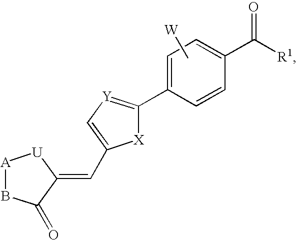
wherein R¹ is hydroxy, NHOR², NHNR³R⁴, or NR⁵OH, wherein R²⁻⁵ are independently hydrogen, lower alkyl, or aryl; W is hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is O, S, or NR⁶, wherein R⁶ is hydrogen or lower alkyl; Y is N, CH, or CR⁷, wherein R⁷ hydrogen or lower alkyl; U is O, S, or NR⁸, wherein R⁸ is hydrogen, lower alkyl, or aryl; and A and B are independently selected from formulas I-VI, 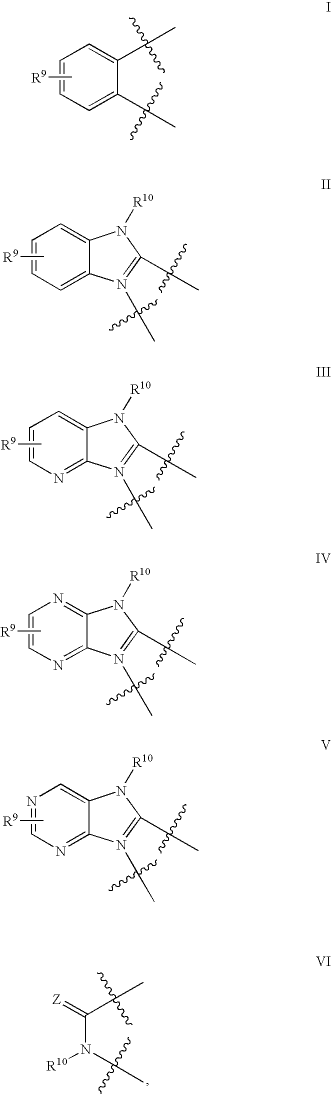
wherein R⁹ is hydrogen, halogen, hydroxy, lower alkyl, alkoxyl, amino, alkylamino, or aryl; and R¹⁰ is hydrogen, lower alkyl, or aryl; Z is O, S, or NR¹¹ wherein R¹¹ is hydrogen, lower alkyl, or aryl; 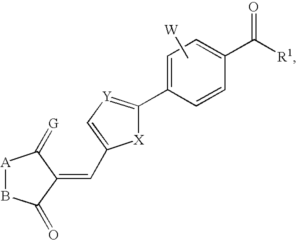
wherein R¹ is hydroxy, NHOR², NHNR³R⁴, or NR⁵OH, wherein R²⁻⁵ are independently hydrogen, lower alkyl, or aryl; W is hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is O, S, or NR⁶, wherein R⁶ is hydrogen or lower alkyl; Y is N, CH, or CR⁷, wherein R⁷ is hydrogen or lower alkyl; G is O, S, or NR⁸, wherein R⁸ is hydrogen, lower alkyl, or aryl; and A and B are independently selected from formulas I-IV, 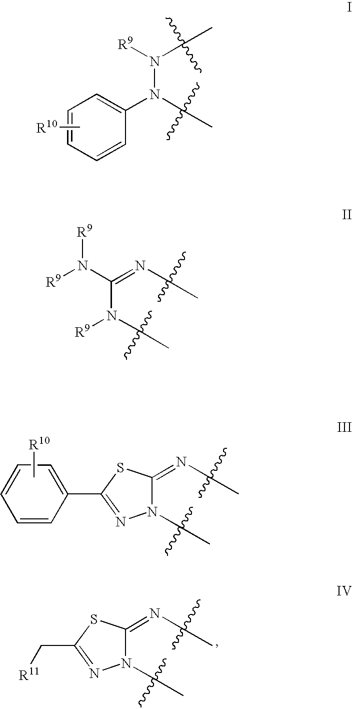
wherein R⁹ is hydrogen, lower alkyl, or aryl; R¹⁰ is hydrogen, alkyl, aralkyl, heteroalkyl, alkene, heteroalkene, alkyne, heteroalkyne, aryl, heteroaryl, alkoxy, halogen, amino, nitro, or alkylamino; and R¹¹ is hydrogen, lower alkyl, aryl, or heteroaryl; 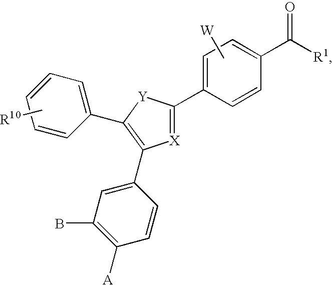
wherein R¹ is OH, NHOR², NHNR³R⁴, or NR⁵OH, wherein R²⁻⁵ are independently hydrogen, lower alkyl, or aryl; W and R¹⁰ are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; X is N, CH, or CR⁷, wherein R⁷ is hydrogen or lower alkyl; Y is O, S, or NR⁶, wherein R⁶ is hydrogen or lower alkyl; R⁸ is hydrogen, lower alkyl, or aryl; and B is hydrogen and A is aryl, heteroaryl, or 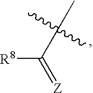
   wherein Z is O, S, NR⁹, NNHR⁹, or NOR⁹, wherein R⁹ is hydrogen, lower alkyl, or aryl, or A and B together are 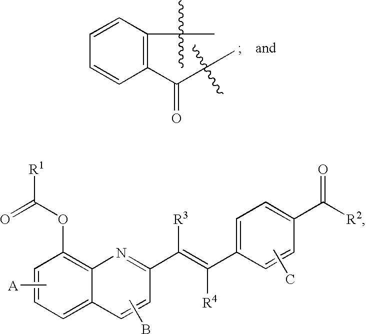
wherein R¹ is alkyl, aryl, or aralkyl; R² is OH, NHOR⁵, NHNR⁶R⁷, or NR⁸OH, wherein R⁵⁻⁸ are independently hydrogen, lower alkyl, or aryl; R³ and R⁴ are hydrogen or alkyl; and A, B, and C are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio.

Examples of the compounds of the invention are shown in Tables 1 and 2. Data illustrating the ability of some of these compounds to inhibit RNase P activity and bacterial growth are provided in Tables 4 and 5. Toxicity data for certain compounds are presented in Table 6.

TABLE 1
Guanylhydrazones

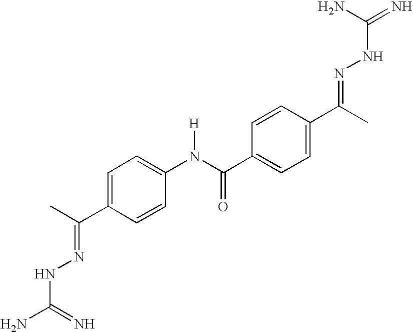

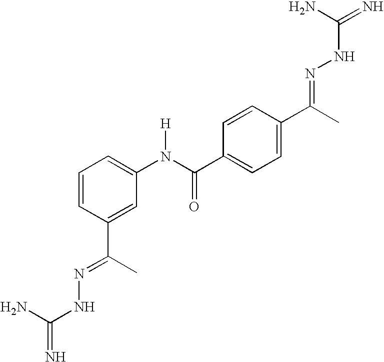

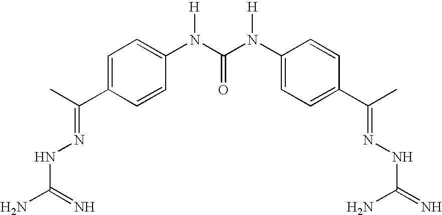

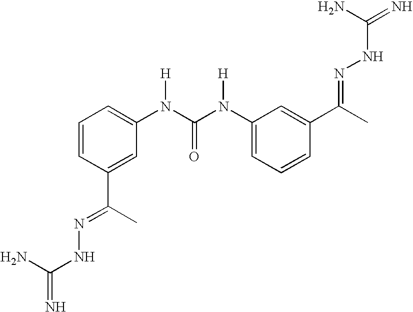

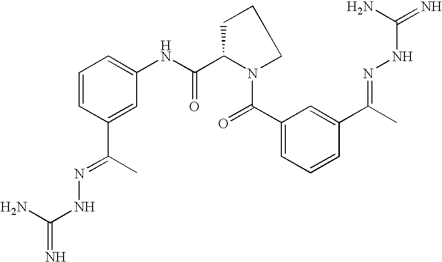

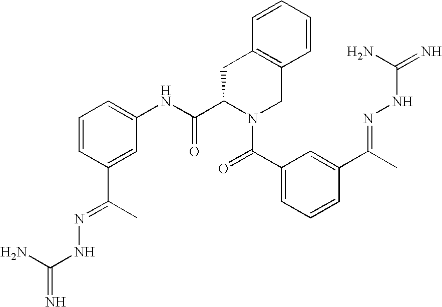

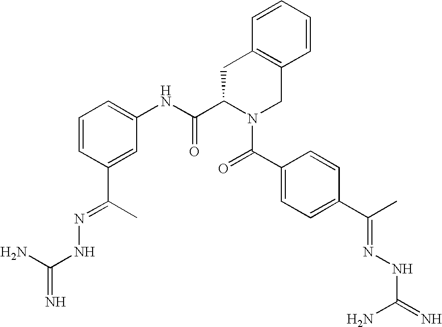

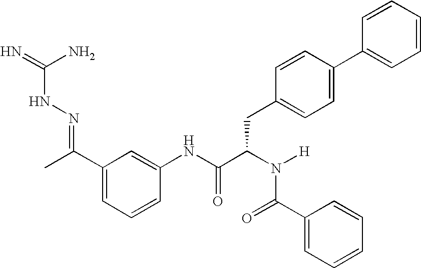

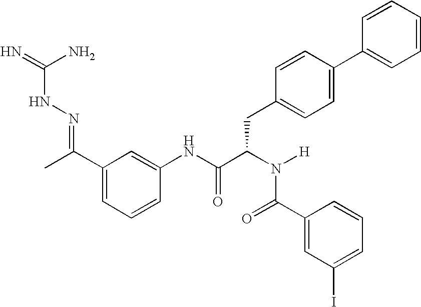

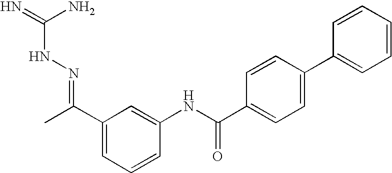

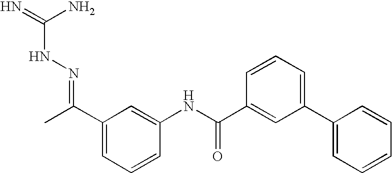

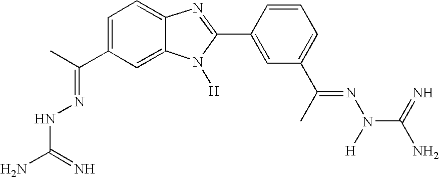

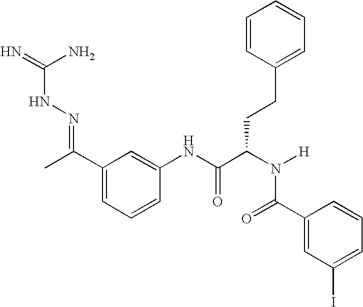

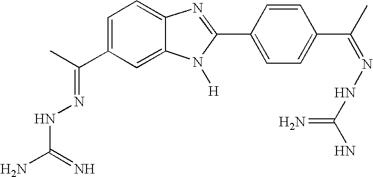

TABLE 2
Benzoic acid compounds

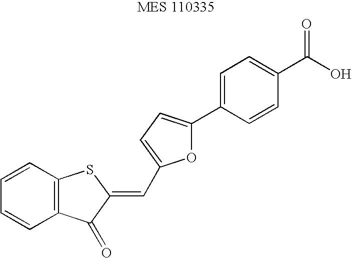

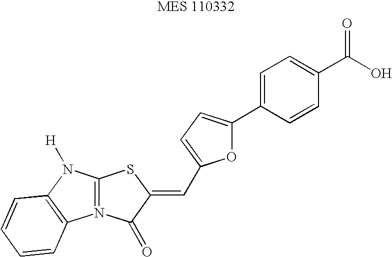

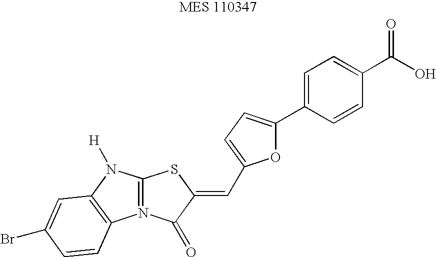

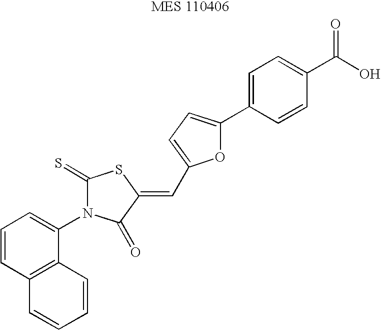

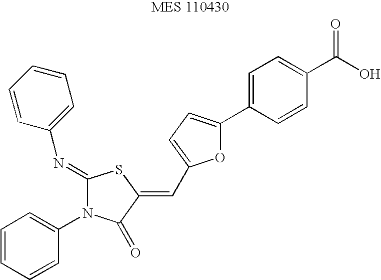

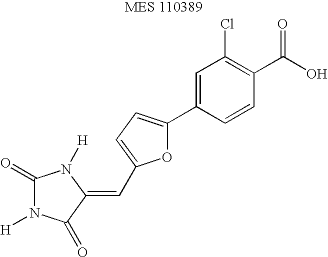

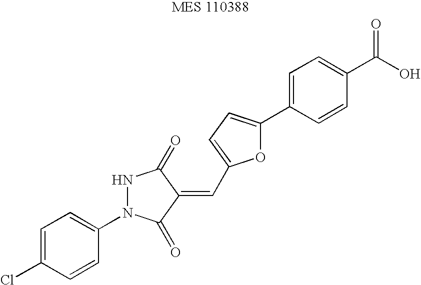

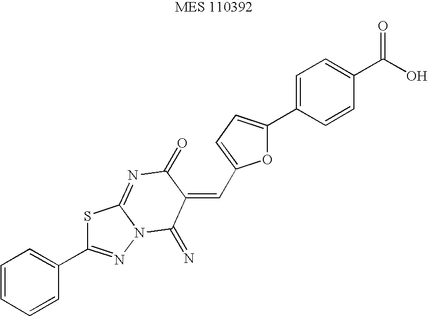

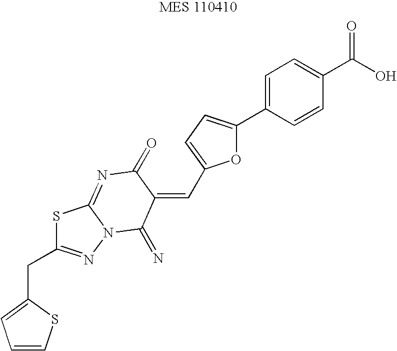

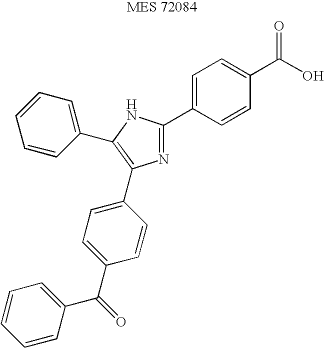

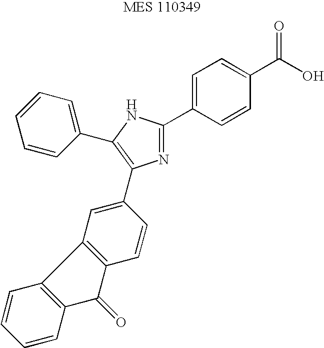

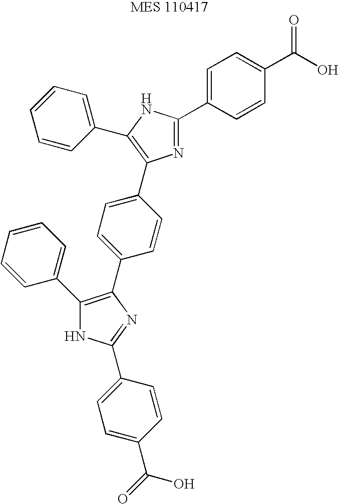

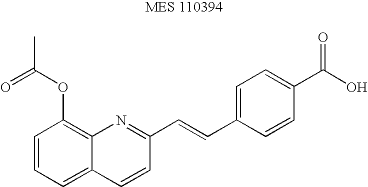

Clinical Applications of RNase P Inhibitors

Compounds which modulate RNase P activity may be administered by any appropriate route for treatment, stabilization, or prevention of a bacterial infection. These compounds may be administered to humans, domestic pets, livestock, or other animals with a pharmaceutically acceptable diluent, carrier, or excipient, in unit dosage form. Administration may be oral, topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or by any other suitable route of administration.

Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in Remington: The Science and Practice of Pharmacy (20th ed., A. R. Gennaro ed., Lippincott: Philadelphia, 2000). Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, and trifluoroacetic acids; polymeric acids such as tannic acid and carboxymethyl cellulose; and inorganic acid such as hydrochloric acid, hydrobromic acid, sulfuric acid, and phosphoric acid. Metal complexes include zinc and iron.

The chemical compounds for use in such therapies may be produced and isolated as described herein or by any standard technique known to those in the field of medicinal chemistry. Conventional pharmaceutical practice may be employed to provide suitable formulations or compositions to administer the identified compound to patients suffering from a condition or at increased risk for a condition involving bacterial infection. Administration may begin before, during, or after the patient has been infected or is symptomatic.

The formulations can be administered to human patients in therapeutically effective amounts (e.g., amounts which prevent, stabilize, eliminate, or reduce a bacterial infection) to provide therapy for a disease or condition associated with a bacterial infection. Typical dose ranges are from about 0.1 μg/kg to about 1 mg/kg of body weight per day. The exemplary dosage of drug to be administered typically depends on such variables as the type and extent of the disorder, the overall health status of the particular patient, the formulation of the compound, and its route of administration. Standard clinical trials may be used to optimize the dose and dosing frequency for any particular compound.

Other Uses of RNase P Inhibitors

Compounds which modulate RNase P activity may also be used for the preservation of food, beverages, cosmetics such as lotions, creams, gels, ointments, soaps, shampoos, conditioners, antiperspirants, deodorants, mouth wash, contact lens products, enzyme formulations, or food ingredients. Methods for use as a preservative include incorporating a compound of the invention into, for example, unpreserved food, beverages, cosmetics, contact lens products, or food ingredients in an amount effective for killing or inhibiting the growth of bacteria.

Thus, a compound of the invention may by useful as a disinfectant, e.g., in the treatment of acne, eye infections, mouth infections, skin infections, or other wounds. It is also contemplated that a compound of the invention is useful for cleaning, disinfecting, or inhibiting bacterial growth on any hard surface. Examples of surfaces which may advantageously be contacted with a compound of the invention are surfaces of process equipment used in dairies, chemical or pharmaceutical process plants, water sanitation systems, paper pulp processing plants, water treatment plants, cooling towers, cooking utensils, hospital operating rooms, or surfaces in any area in which food is prepared (e.g., hospitals, nursing homes, or restaurants). The composition of the invention should be used in an amount which is effective for cleaning, disinfecting, or inhibiting bacterial growth on the relevant surface.

In addition, compounds of the invention are useful for cleaning, disinfecting, or inhibiting bacterial growth on in an in-dwelling device in a patient. In-dwelling devices include, but are not limited to, surgical implants, prosthetic devices, artificial joints, heart valves, pacemakers, vascular grafts, vascular catheters, cerebrospinal fluid shunts, urinary catheters, and continuous ambulatory peritoneal dialysis (CAPD) catheters. A compound of the invention may be used to bathe an in-dwelling device immediately before insertion. The compound will desirably be present, for example, at a concentration of 1 μg/ml to 10 mg/ml for bathing of wounds or indwelling devices. Alternatively, the compound may be administered by injection to achieve a local or systemic effect against relevant bacteria shortly before insertion of an in-dwelling device. Treatment may be continued after surgery during the in-body time of the device.

General Synthetic Strategies

General Description of the Synthesis of Guanylhydrazines and Aryl Guanylhydrazones:

Guanylhydrazines can be prepared from commercially available starting materials as follows. A monoprotected hydrazine (e.g., t-butylcarbazate—Aldrich catalogue number B9,100-5) may be condensed with an aldehyde/ketone and reduced with a hydride reducing agent such as sodium cyanoborohydride to yield a protected monoalkylated hydrazine. Condensation with a suitable guanylating agent such as 1,3-bis(t-butoxycarbonyl)-2-methyl-2-thiopseudourea (Aldrich catalogue number 43,9910-8) or a derivative of this compound (see, for example, Monache et al., J. Med. Chem. 36: 2956, 1993) yields mono- or di-substituted guanylhydrazines, as shown in Scheme 1.

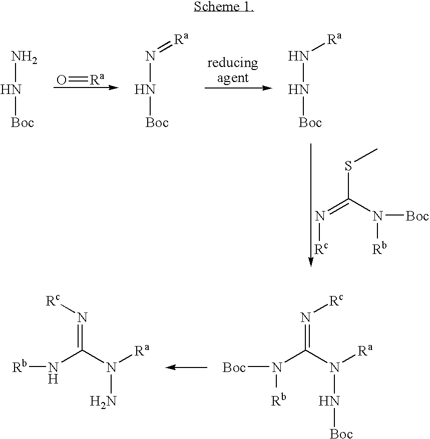

Aryl, e.g., phenyl, biphenyl and naphthyl, guanylhydrazones can be prepared by condensation of guanylhydrazines with aryl aldehydes or ketones as shown in Scheme 2.

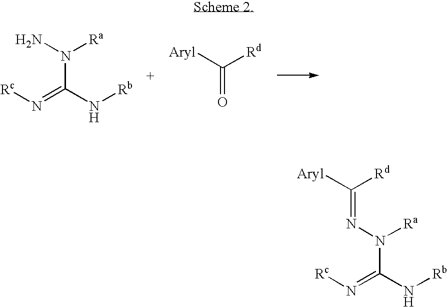

The aryl group may also contain at least one carboxylic acid or amino substituent useful for attachment to other substituents. The reaction is carried out using standard imine condensation techniques (see, for example, J. March, Advanced Organic Chemistry: Reactions, Mechanisms and Structure, Wiley: New York, pp. 896-899, 1992). The condensation reaction may be performed prior to the coupling to another substituent. The guanyl nitrogens of the guanyl hydrazine may be protected (for example, using standard protecting groups, R=Boc or Cbz) or unprotected, during the condensation reaction of Scheme 1.

Alternatively, the bis aldehyde/ketones can be condensed with a monosubstituted hydrazine to generate the bis hydrazones which may be subsequently guanylated employing the reagent described above, as shown in Scheme 3.

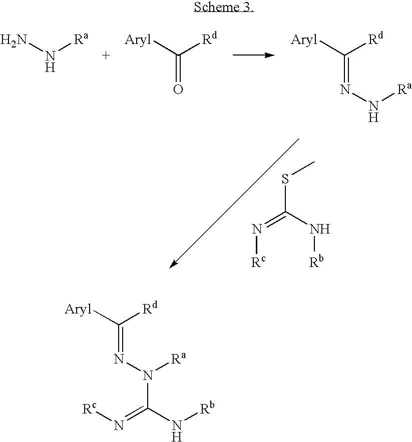

General Description of the Coupling of Precursors:

Coupling of the two components of these species may be accomplished via standard synthetic methods (Schemes 4-6). Amide linkages may be prepared by activation of a carboxylic acid and subsequent reaction with the appropriate amine. Alternatively, addition of a substituted benzylGrignard reagent to an appropriate benzaldehyde would generate the hydroxyethylene linkage moiety. O-alkylation of this species, O-activation and nucleophilic displacement, or dehydration of these species would generate the alkoxyethylene, aminoethylene, and ethenyl linkages. Alternatively, condensation of an appropriate aniline with the substituted benzaldehyde and reduction of the resulting imine would generate the aminomethylene linkage. 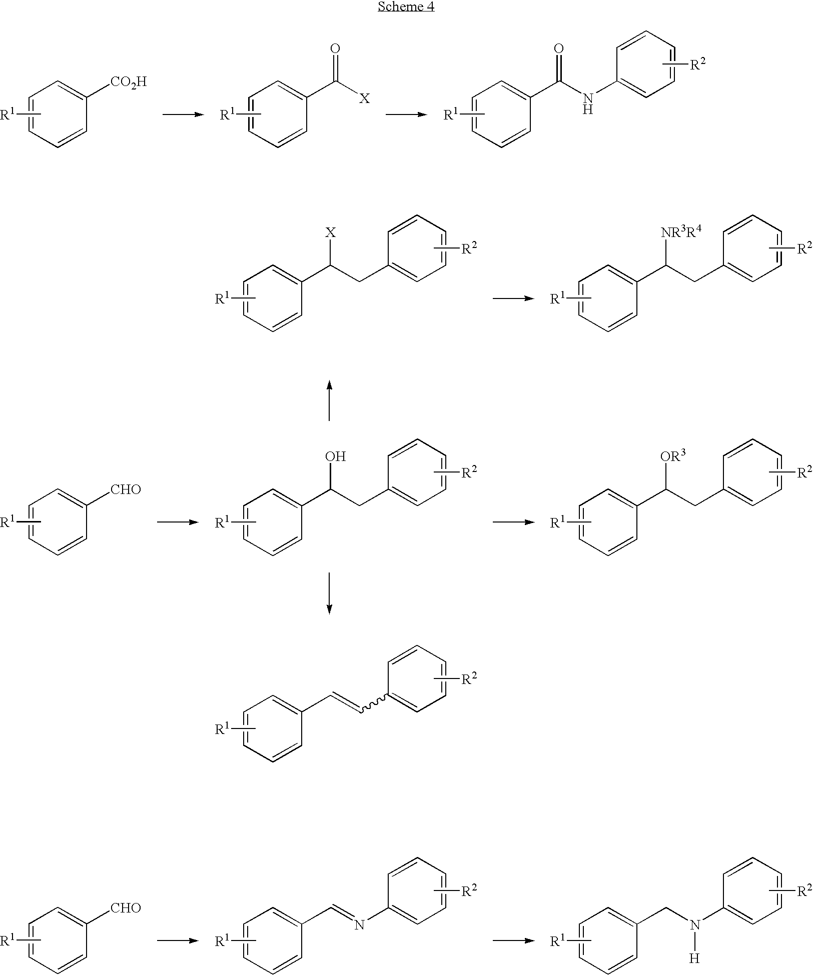
Alternatively, a substituted isocyanate may be coupled with an appropriate aniline to give the urea linked species. 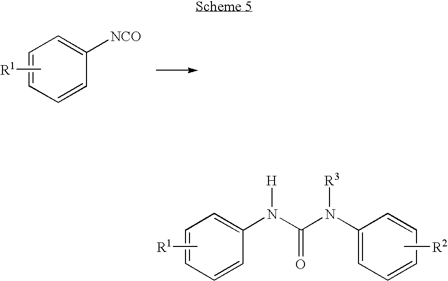
Alternatively, substituted arylnaphthalenes and related species may be prepared by metal catalyzed coupling of the naphthylbromide and an appropriate boronic acid. 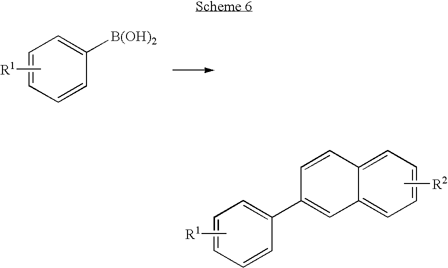
Assays

The ability of compounds of the invention to inhibit RNase P enzymes can be assessed by standard techniques. For example, the cleavage of ptRNA^{Gln} by the enzyme N. Gonorrhea RNase P, can be monitored in the presence and absence of a candidate compound as described in the Example 15. The progress of the RNase P-mediated cleavage reaction can be assessed by measuring the fluorescence polarization level of the TAMRA moiety hybridized to the cleaved substrate.

In addition, the RNase P enzyme activity can also be measured using standard techniques described in the literature (see, e.g., Altman and Kirsebom, Ribonuclease P, The RNA World, 2nd Ed., Cold Spring Harbor Laboratory Press: Cold Spring Harbor, N.Y., 1999; Pascual and Vioque, Proc. Natl. Acad. Sci. 96: 6672, 1999; Geurrier-Takada et al., Cell 35: 849, 1983; Tallsjö and Kirsebom, Nucleic Acids Research 21: 51, 1993; Peck-Miller and Altman, J. Mol. Biol. 221: 1, 1991; Gopalan et al., J. Mol. Biol. 267: 818, 1997; and WO 99/11653).

To screen for compounds that inhibit the activity of the RNase P holoenzymes, compounds are added to a final concentration of 10 μM before the addition of substrate to the sample. A compound is determined to be an inhibitor if it significantly reduces RNase P hydrolysis as compared to the compound-free control sample. Desirably, the compounds identified as inhibitors selectively inhibit the RNase P holoenzymes of one or more pathogenic bacteria without affecting other RNase P holoenzymes. Such inhibitors have the advantage of providing a selective antibacterial treatment that reduces the adverse side effects associated with killing nonpathogenic bacteria. Use of such selective inhibitors also reduces the risk of producing a wide range of resistant bacterial strains.

The ability of compounds of the invention to inhibit bacterial growth can also be assessed by standard testing procedures, such as monitoring bacterial growth in the presence of one or more candidate compounds. Any reduction in bacterial growth, in comparison to an uninhibited control, is a measure of the antibacterial activity of the compound. The antibacterial activity of some compounds of the invention were measured against N. gonorrhea and S. pyogenes, which are representative bacterial species (Example 18).

Other assays that can be used to measure RNase P inhibition are known in the art, for example those described in US Application Publication No. 2003-0134904 A1.

### EXAMPLES

The following examples are merely intended to illustrate various embodiments of the application are not intended to be limiting in any way.

### 1. Bisguanylhydrazone of 4-acetyl-N-(4-acetylphenyl)benzamide (MES 10948)

4-Acetylbenzoic acid (810 mg, 5 mmol) was dissolved in chloroform (20 mL), and oxalyl chloride (1.27 g, 2 eq.) added. Catalytic DMF (4 drops) was added, and the mixture stirred for 2 hr at r.t. The volatiles were removed under a stream of nitrogen, and the residue was dried under vacuum. The crude acid chloride was dissolved in chloroform (20 mL), and to this added 4-aminoacetophenone (675 mg, 1 eq.) and diisopropylethylamine (1.74 mL, ˜2 eq.). The mixture was then stirred overnight at r.t. The reaction mixture was quenched with saturated aqueous sodium bicarbonate solution (25 mL), diluted with chloroform (20 mL), and the organic phase was separated. The organic phase was washed with saturated aqueous sodium bicarbonate solution (3×30 mL), water (30 mL), 1N hydrochloric acid (3×30 mL), and brine (30 mL) before drying over sodium sulfate. Evaporation of the solvent gave a yellow solid that was recrystallized from hot chloroform to give 4-acetyl-N-(4-acetylphenyl)benzamide.

4-Acetyl-N-(4-acetylphenyl)benzamide (14 mg, 0.05 mmol) was dissolved in dry DMSO (250 μL). To this solution was added dry ethanol (160 μL), aminoguanidine hydrochloride (3 eq., 18 mg) and ethanolic hydrochloride acid (80 μL of a 99:1 mixture of ethanol:conc. hydrochloric acid). The mixture was heated to 105° C. in a sealed vial for 5 days. The ethanol was allowed to evaporate at ˜95° C. for 1 hr, and the remaining volatiles were evaporated under a stream of argon for 5 min. The crude DMSO solution of the product was purified by preparative reverse-phase HPLC employing 20/80 acetonitrile/water (both 0.1% trifluoroacetic acid) as mobile phase. The product fractions were evaporated on a rotary evaporator to remove the acetonitrile and then frozen and lyophilyzed to give the bis trifluoroacetate salt of the title product as a white feathery solid (16 mg). ¹H NMR (DMSO) δ=10.60 (s, 1H), 10.48 (s, 1H), 10.45 (s, 1H), 8.14 (d, J=8.3 Hz, 2H), 8.02 (d, J=8.1 Hz, 2H), 8.00 (d, J=6.3 Hz, 2H), 7.87 (d, J=8.9 Hz, 2H), 7.72 (br s, 4H), 7.61 (br s, 4H), 2.36 (s, 3H), 2.30 (s, 3H) ppm.

### 2. Bisguanylhydrazone of 4-acetyl-N-(3-acetylphenyl)benzamide (MES 10648)

The bis trifluoroacetate salt of the title compound may be prepared in a manner identical to Example 1 except employing 3-aminoacetophenone instead of 4-aminoacetophenone. ¹H NMR (DMSO) δ=10.61 (s, 1H), 10.50 (s, 1H), 10.47 (s, 1H), 8.13 (m, 2H), 8.04 (m, 4H), 7.90 (m, 2H), 7.73 (br s, 4H), 7.65 (br s, 4H), 2.38 (s, 3H), 2.29 (s, 3H) ppm.

### 3. Bisguanylhydrazone of N,N′-bis(4-acetylphenyl)urea (MES 10950):

4-Aminoacetophenone (135 mg, 1 mmol) was dissolved in methylene chloride (5 mL), and carbonyldiimidazole (0.5 eq., 81 mg) was added. The mixture was stirred for 3 days at r.t. The resultant solid was collected by filtration, washed with ethyl acetate (2 mL), and dried under vacuum to give N,N,-bis(4-acetylphenyl)urea (76 mg). N,N′-bis(4-acetylphenyl)urea (15 mg, 0.05 mmol) was converted to the bisguanylhydrazone in a manner identical to Example 1, to give the bis trifluoroacetate salt of the title compound (6 mg) as a mixture of rotamers. Major rotamer: ¹H NMR (DMSO) δ=10.50 (s, 1H), 10.48 (s, 1H), 9.80 (s, 1H), 9.27 (s, 1H), 7.91 (m, 4H), 7.61 (br s, 8H), 7.53 (m, 4H), 7.35 (m, 2H), 2.28 (s, 3H), 2.26 (s, 3H) ppm.

### 4. Bisguanylhydrazone of N,N-bis(3-acetylphenyl)urea (MES 10949):

The bis trifluoroacetate salt of the title compound may be prepared in a manner identical to Example 3 except employing 3-aminoacetophenone instead of 4-aminoacetophenone. Major rotamer ¹H NMR (DMSO) δ=10.55 (s, 1H), 10.52 (s, 1H), 9.62 (s, 1H), 8.95 (s, 1H), 8.06 (s, 2H), 7.9-7.5 (m, 12H), 7.35 (m, 2H), 2.30 (s, 3H), 2.27 (s, 3H) ppm.

### 5. Bisguanylhydrazone of (S)-1-(3-acetylbenzoyl)-N-(3-acetylphenyl) prolinamide (MES 10926)

(S)-N-Boc-proline (215 mg, 1 mmol) and 3-aminoacetophenone (135 mg, 1 eq.) were dissolved in dry DMF (4 mL). To this solution was added BOP (442 mg) and diisopropylethylamine (560 μL), and the resulting solution stirred at r.t for 24 hr. The reaction was quenched by the addition of 0.2 N aqueous sodium hydroxide (50 mL), and the mixture stirred for 4 hr. The reaction mixture was extracted with ethyl acetate (50 mL), and the organic phase washed with 1 N aqueous sodium hydroxide (25 mL), 1 N hydrochloric acid (25 mL), water (3×50 mL), brine (50 mL), and dried over sodium sulfate. Evaporation of the solvent gave (S)-N-Boc-(3-acetylphenyl)prolinamide as a white foam (243 mg).

(S)-N-Boc-(3-acetylphenyl)prolinamide (83 mg, 0.25 mmol) was dissolved in methylene chloride (0.5 mL), and to this added trifluoroacetic acid (0.5 mL). The mixture was stirred at r.t for 45 min, and then the volatiles were removed on a rotary evaporator, and the resultant oil dried under vacuum. The deprotected prolinamide was dissolved in dry DMF (1 mL). To this solution was added BOP (111 mg) and triethylamine (190 μL), and 3-acetylbenzoic acid (42 mg), and the resulting solution stirred at r.t for 24 hr. The reaction was quenched by the addition of 0.2 N aqueous sodium hydroxide (15 mL), and the mixture stirred for 4 hr. The resulting solid was collected by filtration, washed with water (3×5 mL) and dried under vacuum to give 1-(3-acetylbenzoyl)-N-(3-acetylphenyl)prolinamide (67 mg), which was converted to the bisguanylhydrazone in a manner identical to Example 1. The crude product was purified by preparative reverse-phase HPLC employing 18/82 acetonitrile/water (both 0.1% trifluoroacetic acid) as mobile phase. The product fractions were evaporated on a rotary evaporator to remove the acetonitrile and then frozen and lyophilyzed to give the bis trifluoroacetate salt of the title product as a white feathery solid (16 mg). ¹H NMR (DMSO) δ=10.60 (s, 1H), 10.58 (s, 1H), 10.23 (s, 1H), 8.05 (m, 3H), 7.70 (m, 11H), 7.38 (m, 2H), 4.64 (m, 1H), 3.51 (m, 2H), 2.34 (s, 3H), 2.30 (s, 3H), 1.93 (m, 4H) ppm.

### 6. Bisguanylhydrazone of (S)-2-(3-acetylbenzoyl)-N-(3-acetylphenyl)tetrahydro-isoquinoline-3-carboxamide (MES 10848)

The bis trifluoroacetate salt of the title compound was prepared in an analogous manner to Example 5 except employing (S)-tetrahydroisoquinoline-3-carboxylic acid. ¹H NMR (DMSO) δ=10.62 (s, 1H), 10.57 (s, 1H), 10.34 (s, 1H), 8.2-7.1 (m, 20H), 5.01 (m, 1H), 4.80 (m, 1H), 4.61 (m, 1H), 3.18 (m, 2H), 2.34 (s, 3H), 2.28 (s, 3H) ppm.

### 7. Bisguanylhydrazone of (S)-1-(4-acetylbenzoyl)-N-(3-acetylphenyl) tetrahydro-isoquinoline-3-carboxamide (MES 10946)

The bis trifluoroacetate salt of the title compound was prepared in an analogous manner to Example 6 employing 4-acetylbenzoic acid in place of 3-acetylbenzoic acid. ¹H NMR (DMSO) δ=10.63 (s, 1H), 10.54 (s, 1H), 10.33 (s, 1H), 8.14-7.09 (m, 20H), 4.98 (m, 1H), 4.60 (m, 2H), 3.10 (m, 2H), 2.36 (s, 3H), 2.28 (s, 3H) ppm.

### 8. Guanylhydrazone of (S)-1-(3-iodobenzoyl)-N-(3-acetylphenyl)biphenyl-alaninamide (MES 10908)

(S)-1-(3-iodobenzoyl)-N-(3-acetylphenyl)biphenyl-alaninamide was prepared in an analogous manner to (S)-1-(3-acetylbenzoyl)-N-(3-acetylphenyl)prolinamide from (S)-N-Boc-biphenylalanine. The title guanylhydrazone was prepared in an analogous manner to Example 1 with aminoguanidine hydrochloride (12 mg, 2 eq.) and purified by preparative reverse-phase HPLC employing 45/55 acetonitrile/water (both 0.1% trifluoroacetic acid) as mobile phase. The product fractions were evaporated on a rotary evaporator to remove the acetonitrile and then frozen and lyophilyzed to give the bis trifluoroacetate salt of the title product as a white feathery solid (10 mg). ¹H NMR (DMSO) δ=10.58 (s, 1H), 10.40 (s,1H), 8.87 (d, 7.8 Hz, 1H), 8.23 (s, 1H), 7.99 (s, 1H), 7.90 (d, J=8.0 Hz, 1 Hz), 7.85 (d, J=7.8 Hz, 1H), 7.76 (m, 2H), 7.62-7.25 (m, 15H), 4.86 (m, 1H), 3.15 (m, 2H), 2.29 (s, 3H) ppm.

### 9. Guanylhydrazone of (S)-1-benzoyl-N-(3-acetylphenyl) biphenylalaninamide (MES 10910)

The title compound was prepared in a manner analogous to Example 8 and purified by preparative reverse-phase HPLC employing 40/60 acetonitrile/water (both 0.1% trifluoroacetic acid) as mobile phase. The product fractions were evaporated on a rotary evaporator to remove the acetonitrile and then frozen and lyophilyzed to give the bis trifluoroacetate salt of the title product as a white feathery solid (15 mg). ¹H NMR (DMSO) δ=10.54 (s, 1H), 10.33 (s, 1H), 8.78 (d, J=8.3 Hz, 1H), 8.00 (s, 1H), 7.86 (m, 2H), 7.75 (m, 2 Hz), 7.8-7.3 (m, 17H), 4.90 (m, 1H), 3.13 (m, 2H), 2.29 (s, 3H) ppm.

### 10. Guanylhydrazone of (S)-1-(3-iodobenzoyl)-N-(3-acetylphenyl) homophenyl-alaninamide (MES 10914)

(S)-1-(3-iodobenzoyl)-N-(3-acetylphenyl)homophenyl-alaninamide was prepared in an analogous manner to (S)-1-(3-acetylbenzoyl)-N-(3-acetylphenyl)prolinamide from (S)-N-Boc-homophenylalanine. The title guanylhydrazone was prepared in an analogous manner to Example 1 with aminoguanidine hydrochloride (12 mg, 2 eq.) and purified by preparative reverse-phase HPLC employing 50/50 acetonitrile/water (both 0.1% trifluoroacetic acid) as mobile phase. The product fractions were evaporated on a rotary evaporator to remove the acetonitrile and then frozen and lyophilized to give the bis trifluoroacetate salt of the title product as a white feathery solid (8 mg). ¹H NMR (DMSO) δ=10.60 (s, 1H), 10.21 (s, 1H), 8.82 (d, 7.3 Hz, 1H), 8.30 (s, 1H), 8.00 (s, 1H), 7.93 (m, 2H), 7.72 (m, 2H), 7.63 (br s, 4H), 7.29 (m, 7H), 4.60 (m, 1H), 2.75 (m, 1H), 2.66 (m, 1H), 2.28 (s, 3H), 2.15 (m, 2H) ppm.

### 11. Guanylhydrazone of N-(3-acetylphenyl)-4-biphenylcarboxamide (MES 10938)

3-Aminoacetophenone (40 mg, 0.3 mmol) and 4-biphenylcarboxylic acid (59 mg, 1 eq.) were dissolved in dry DMF (1.25 mL), and to this solution was added BOP (133 mg) and triethylamine (166 μL). The mixture was stirred at r.t. for 24 hr and then quenched by the addition of 0.2 N aqueous sodium hydroxide (15 mL). After stirring for 4 hr, the tan precipitate was isolated by filtration, washed with water (3×10 mL) and dried under vacuum to give N-(3-acetylphenyl)-4-biphenylcarboxamide (58 mg).

N-(3-acetylphenyl)-4-biphenylcarboxamide was converted to the guanyl-hydrazone in manner identical to Example 8 to give the bis trifluoroacetate salt of the title compound as a white feathery solid (12 mg). ¹H NMR (DMSO) δ=10.57 (s, 1H), 10.36 (s, 1H), 8.22 (s, 1H), 8.09 (d, J=8.3 Hz, 2H), 7.88 (d, J=6.4 Hz, 1H), 7.86 (d, J=8.3 Hz, 2H), 7.80(d, J=7.9 Hz, 1H), 7.77 (d, J=7.3 Hz, 2H), 7.63 (br s, 4H), 7.53 (t, J=7.7 Hz, 2H), 7.44, (m, 2H), 2.32 (s, 3H) ppm.

### 12. Guanylhydrazone of N-(3-acetylphenyl)-3-biphenylcarboxamide (MES 10915)

The bis trifluoroacetate salt of the title compound was prepared in a manner identical to Example 11 except employing 3-biphenylcarboxylic acid, and purified by preparative reverse-phase HPLC employing 38/62 acetonitrile/water (both 0.1% trifluoroacetic acid) as mobile phase. The product fractions were evaporated on a rotary evaporator to remove the acetonitrile and then frozen and lyophilyzed to give the bis trifluoroacetate salt of the title product as a white feathery solid (6 mg). ¹H NMR (DMSO) δ=10.62 (s, 1H), 10.42 (s, 1H), 8.25 (s, 1H), 8.20 (s, 1H), 7.97 (d, J=7.8 Hz, 1 Hz), 7.90 (m, 2H), 7.80 (m, 3H), 7.65 (m, 5H), 7.53 (m, 2H), 7.44 (m, 2H), 2.33 (s, 3H) ppm.

### 13. Bisguanylhydrazone of 2-(4-acetylphenyl)-5-acetylbenzimidazole (MES 10963)

4-Acetyl-N-(4-acetylphenyl)benzamide (282 mg, 1 mmol), as prepared for Example 1 (MES 10948) was slowly added to fuming nitric acid (4 mL) at −5° C., over 30 min. After addition was complete, the resulting solution was stirred at −5° C. for 20 min and then quenched by pouring onto ice (100 g). The resulting yellow solid was collected by filtration, washed with water (2×25 mL) and dried under vacuum. The crude product was recrystalized from hot ethyl acetate to give 4-acetyl-N-(4-acetyl-2-nitrophenyl)benzamide (200 mg).

4-Acetyl-N-(4-acetyl-2-nitrophenyl)benzamide (130 mg, 0.4 mmol) was dissolved in ethanol (2 mL). To this solution was added ammonium formate (12 eq, 302 mg), water (1 μL) and platinum (IV) oxide (5 mg), and the mixture stirred at rt for 10 days. The reaction was quenched by diluting with ethyl acetate/methanol (4:1) (125 mL) and washed with 0.2 N aqueous sodium hydroxide (100 mL), water (100 mL) and then dried over sodium sulfate. Evaporation of the solvent gave a crude product which was crystallized from ethyl acetate/hexanes (1:1) (20 mL) to give 4-acetyl-N-(4-acetyl-2-aminophenyl)benzamide (66 mg). An addition aliquot of product could be recovered from the mother liquors.

4-Acetyl-N-(4-acetyl-2-aminophenyl)benzamide (11 mg, 0.037 mmol) was dissolved in dry DMSO (188 μL). To this solution was added dry ethanol (120 μL), aminoguanidine hydrochloride (3 eq., 13.5 mg) and ethanolic hydrochloride acid (60 μL of a 99:1 mixture of ethanol:conc. hydrochloric acid). The mixture was heated to 105° C. in a sealed vial for 5 days. After 5 days a yellow crystalline solid had separated from the reaction mixture, this solid was recovered by filtration, washed with ethanol (2×250 μL), and dried under vacuum to give the title compound as the hydrochloride salt (11 mg). ¹HNMR (DMSO) δ=11.30 (s, 1H), 11.18 (s, 1H), 8.37 (d, J=8.6 Hz, 2H), 8.26 (d, J=8.6 Hz, 2H), 8.17 (s, 1H), 8.15 (d, J=9.1 Hz, 1H), 7.85 (m, 8H), 7.73 (d, J=8.8 Hz, 1H), 2.43 (s, 3H), 2.37 (s, 3H) ppm.

### 14. Other Compounds

The compounds listed in Table 3 were obtained from commercial sources (Specs, Columbia, Md. or ChemBridge, San Diego, Calif.). Alternatively, the compounds could be synthesized by methods known in the art.

TABLE 3
Sources for selected compounds.

| Compound | Source | Inventory # |
|---|---|---|
| MES 72084 | Specs | AG-205/11945023 |
| MES 110332 | ChemBridge | 5376464 |
| MES 110335 | ChemBridge | 5377260 |
| MES 110347 | ChemBridge | 5677712 |
| MES 110349 | ChemBridge | 5686056 |
| MES 110388 | ChemBridge | 6170904 |
| MES 110389 | ChemBridge | 6223549 |
| MES 110392 | ChemBridge | 6371810 |
| MES 110394 | ChemBridge | 6375115 |
| MES 110406 | ChemBridge | 6800800 |
| MES 110410 | ChemBridge | 7377010 |
| MES 110417 | ChemBridge | 8025745 |
| MES 110430 | ChemBridge | 8080717 |

### Example 15

### Time Resolved Fluorescence RNase P Inhibition Assay

96-well Nunc MaxiSorp FluoroNunc plates were coated with 50 μl/well of 1 μg/ml of streptavidin in base buffer (150 mM KCl, 5 mM MgCl₂, 50 mM Tris, pH 7.6) and incubated overnight. The plates were washed using TRF.96 protocol, and then blocked with 150 μl of a 1 mg/ml BSA solution in base buffer and incubated with shaking for sixty minutes. The plates were then washed twice with base buffer plus 0.01% Tween20. 50 μl of 40 nM T17-bt (biotinylated DNA oligonucleotide complementary to the RNaseP substrate leader sequence used to capture intact RNaseP substrate) was then added to all wells, and the plates were incubated with shaking for 1 hr.

A 0.4 nM solution of N. gonorrhea M1 RNA was made from a 1 μM stock solution. A 0.5 nM solution of N. gonorrhea C5 protein is made by diluting a 10 μM stock. ΔUL-ptGLN UTP-bt (10 μM stock) was diluted into PA buffer for a concentration of 40 nM.

Compounds were serially diluted in DMSO from 20 mM stocks to 6, 2, 0.6, 0.2, and 0.06 mM dilutions. These titrations were then diluted (2 μl to 100) in PA buffer, and 12.5 μl of each concentration was added in duplicate to a 96-well V-bottom polypropylene plate. The highest concentration was put in row G and the lowest in row B. Rows A & H received 12.5 μl of DMSO/PA buffer.

The enzyme reaction was initiated in the V-bottom polypropylene plates containing compound dilutions by adding 12.5 μl of the N. gonorrhea M1 solution to rows A-G. PA buffer was added to row H. 12.5 μl of the C5 solution was added similarly to the M1 solutions. 12.5 μl of the substrate solution was then added to all wells. The plates were incubated for 30 minutes at which time 12.5 μl of stop buffer was added to all wells. (Stop buffer: 500 μg/ml polyC/50 nM SA-Eu in 335 mM MgCl₂ PA buffer).

The T17-biotin-coated plate was washed three times, and 37.5 μl of 75 mM MgCl₂ PA buffer was added to each well. After 30 minutes incubation with the stop buffer, 12.5 μl from all of the enzyme reaction wells was transferred to the T17 plates. The plates were then incubated for 1.5 hr at room temperature with shaking. The plates were washed three times and 40 μl of Delfia enhancement solution (Wallac Oy) was added to all wells. The plates were then read on the Victor 2 plate reader.

### Example 16

### RNase P Inhibition Polyacrylamide Gel Assay

The substrate, the precursor of tRNA^{Gln}, (pGln), from Synechocystis (Pascual and Vioque, 1999), was synthesized in vitro from the corresponding cDNA by T7 RNA polymerase in the presence of α³²P-GTP. Each control RNase P reaction of 10 μl contained 50 mM Tris-HCl (pH 7.8), 10 mM MgCl₂, 100 mM NH₄Cl, 1 mM dithiothreitol, and 0.4-1.0 pmol pGln substrate, radiolabeled to a specific activity of 1000-10,000 cpm/pmol. The reaction mixture, containing 0.1-1 nM holoenzyme, was incubated for 5-60 min at room temperature (18-24° C.), and the reaction was terminated by addition of an equal volume of 40 mM EDTA/8 M urea. The samples were electrophoresed in denaturing 8% polyacrylamide gels. The activity was quantified by exposure of the gel to a phosphorimaging screen. For test reactions that require the addition of compounds, the RNA subunits were pre-incubated with compound for 10 min at room temperature in the presence of buffer, followed by addition of the protein subunit. After a further 10 minutes of incubation, the radiolabeled substrate was added, and the reaction initiated. Products were analyzed as just described. The IC₅₀ was the concentration of compound that was required to inhibit RNase P activity by 50%.

TABLE 4
In vitro efficacy of selected compounds

| | | N. gonorrhoeae |
|---|---|---|
| | N. gonorrhoeae | Gel assay |
| | TRF IC₅₀(μM) | IC₅₀(μM) |
| Example | (Example 15) | (Example 16) |
| MES 10948 | 2 | 1 |
| MES 10648 | 5 | 2 |
| MES 10950 | 6 | 7 |
| MES 10949 | 6 | 30 |
| MES 10926 | 43 | 100 |
| MES 10848 | 3 | 6 |
| MES 10908 | 5 | 6 |
| MES 10948 | 3 | 2 |
| MES 10910 | 7 | n/a |
| MES 10914 | 6 | n/a |
| MES 10938 | 80 | n/a |
| MES 10915 | 16 | 10 |
| MES 110392 | 2 | — |
| MES 110394 | 9 | — |
| MES 110410 | 15 | — |
| MES 110417 | 10 | — |
| MES 110430 | 7 | — |

### 17. Bacterial Inhibition Assay:

Compounds of the invention (see Table 5) were assayed for their ability to inhibit bacterial growth. Compounds were diluted from 10 mM DMSO stocks to 3 mM and 1 mM in DMSO. The compounds were further diluted from these stocks into saline for 200, 120, and 20 μM stocks. Control antibiotics were diluted similarly. Overnight cultures of bacteria were made in the following manner. N. gonorrhea was streaked onto a chocolate agar plate and incubated at 35° C./5% CO₂. Rather than make an overnight culture of S. pyogenes, a loopful of S. pyogenes from a blood plate or a stock plate was used the following day for direct cell suspension.

On the following day bacteria were prepared by dilution into saline with O.D. 625 nM readings taken to determine the concentration of the bacteria. CFUs (colony forming units) were determined using the formula: CFU/mL=OD₆₂₅×(1.5×10⁸ CFU/mL/OD McFarland std)×dilution. The four bacterial cultures were diluted initially to 5.5×10⁷ CFU/mL. The bacteria were then further diluted into medium to 5.5×10⁵ CFU/mL for S. pyogenes and 5.5×10⁶ CFU/mL for N. gonorrhea. S. pyogenes and N. gonorrhea were grown in CAMHB-3% LHB medium. The bacteria were added one per plate at 200 μL per well. Compounds were added in 10 μL aliquots for final concentrations of 10, 3, and 1 μM in duplicate. Control antibiotics, penicillin for S. pyogenes and ciprofloxacin for N. gonorrhea, were added from 0.8 mg/ml to 0.003 mg/ml. Plates were incubated at 35° C. with O₂ for 16-20 hours for S. pyogenes, and read at OD₆₆₅ in a Victor2 plate reader. Plates were incubated at 35° C. with 5% CO₂ for 24 hours for N. gonorrhea at which time 40 μL of MTS reagent is added per well and incubated for 1 hour in same incubator. The plates were read at OD₄₉₀. Compounds were tested at 1 μM, 3 μM, and 10 μM concentrations. The results, which are expressed as a percentage of the control, were calculated using equation 2. In this equation, O.D. is optical density; (O.D. compounds+bacteria) is the optical density observed for bacteria grown in the presence of a compound of the invention; (O.D. blank) is the optical density in the absence of bacteria; and (O.D. bacteria) is the optical density observed for bacteria growing uninhibited. The assay results are provided in Table 5. Other RNase P inhibitors may be tested similarly using any bacteria of interest.

$$\% control = ( \frac{( O . D . compound + bacteria ) - ( O . D . blank )}{( O . D . bacteria ) - ( O . D . blank )} ) \times 100. \qquad Eq 2$$

TABLE 5
Antibacterial efficacy of selected compounds

| | N. gonorrhoeae | S. pyogenes |
|---|---|---|
| Compound | MIC(μM) | MIC(μM) |
| MES 10948 | 3 | 0.3 |
| MES 10648 | 10 | 1 |
| MES 10950 | 30 | 10 |
| MES 10949 | 100 | 30 |
| MES 10926 | 80 | 30 |
| MES 10848 | 100 | 10 |
| MES 10908 | >100 | 10 |
| MES 110392 | 100 | >100 |
| MES 110394 | 10 | >100− |
| MES 110410 | 30 | >100 |
| MES 110417 | 30 | >100 |
| MES 110430 | 30 | >100 |

### 18. Toxicity Assay

Compounds of the invention (see Table 6) were assayed for cellular toxicity as follows. Whole blood was drawn from a volunteer, and the red cells were separated from the buffy coat cells by centrifugation over ficoll-paque. The resulting peripheral blood mononuclear cells (PBMC) were collected from the interface and washed extensively with PBS by centrifugation to remove platelets and cellular debris. The cells were then plated in 96-well tissue culture plates at a density of 5×10⁵ cells per mL at 200 μL per well. After an hour incubation, the candidate compounds were added at the appropriate concentrations diluted from DMSO stocks into assay buffer (RPMI medium supplemented with 10% FCS). The cells were incubated at 37° C., 100% humidity and 5% CO₂ for 24 hours at which time MTS reagent was added per the manufacturer's (Promega) instructions. After 2-3 hours incubation the optical density of the wells was read on a spectrophotometer. Viable cells turn the MTS reagent from a yellow solution to a blue solution but dead cells do not. The data are evaluated using equation 2 as described in Example 17, where bacterial cells are replaced by PBMC cells in the measurements. Representative assay results are provided in Table 4. The data describes the toxicity of these compounds to a representative human cell population (PBMC's). This toxicity data can be compared to the activity in the bacterial growth assays, and used to identify compounds that selectively inhibit bacterial cell growth without adversely effecting eukaryotic cell types such as PBMC's.

TABLE 6
Cellular toxicity of selected compounds

| Compound | PBMC's TC₅₀(μM) |
|---|---|
| MES 10948 | >100 |
| MES 10648 | >100 |
| MES 10950 | >100 |
| MES 10949 | >100 |
| MES 10926 | >100 |
| MES 10848 | >100 |
| MES 10908 | 5 |

### Other Embodiments

All publications, patent applications, and patents referenced in this specification are hereby incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are in the claims.

1. A compound of the formula: 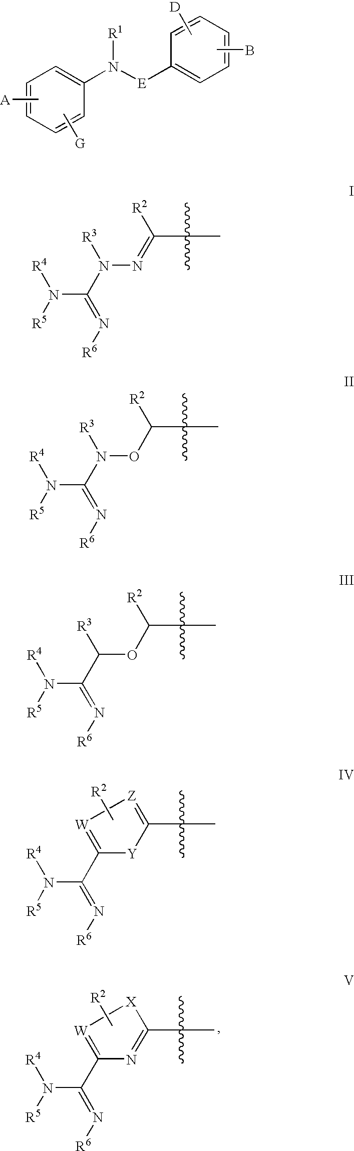
wherein A and B are independently selected from formulas I-V; D and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; E is C═O, C═S, C═CR⁸R⁹, or C═NR⁷; R¹⁻⁹ are independently hydrogen, alkyl, aryl, or aralkyl; W and Z are independently CH, C-alkyl, or N; and X and Y are independently NH, N-alkyl, O, or S.

2. The compound of claim 1, wherein A and B are formula I.

3. The compound of claim 2, having the formula:

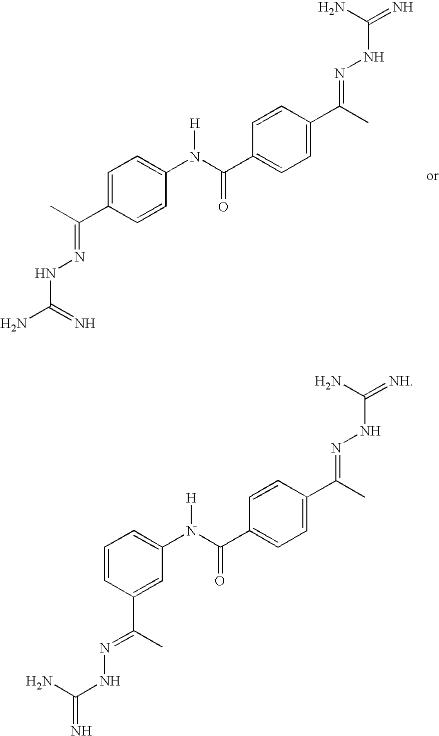

4. The compound of claim 1, wherein E is C═O.

5.-18. (canceled)

19. A compound of the formula: 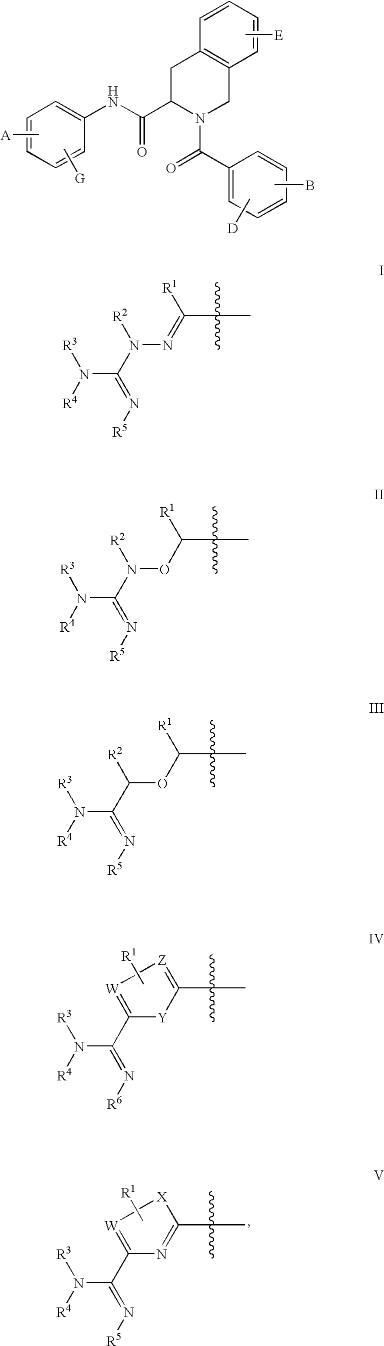
wherein A and B are independently selected from formulas I-V; D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; R¹⁻⁶ are independently hydrogen, alkyl, aryl, or aralkyl; W and Z are independently CH, C-alkyl, or N; and X and Y are independently NH, N-alkyl, O, or S.

20. The compound of claim 0 having the formula:

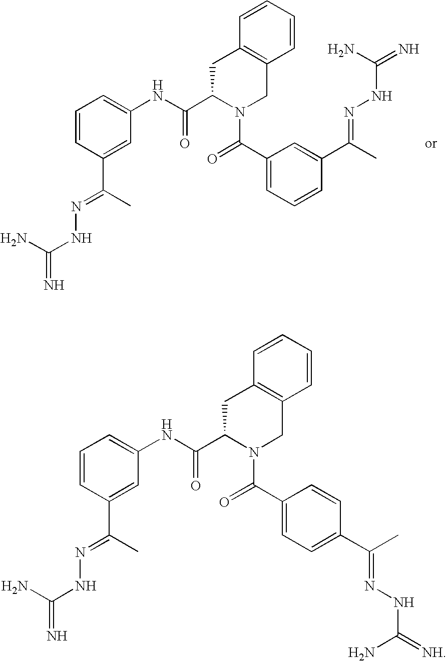

21.-22. (canceled)

23. A compound of the formula: 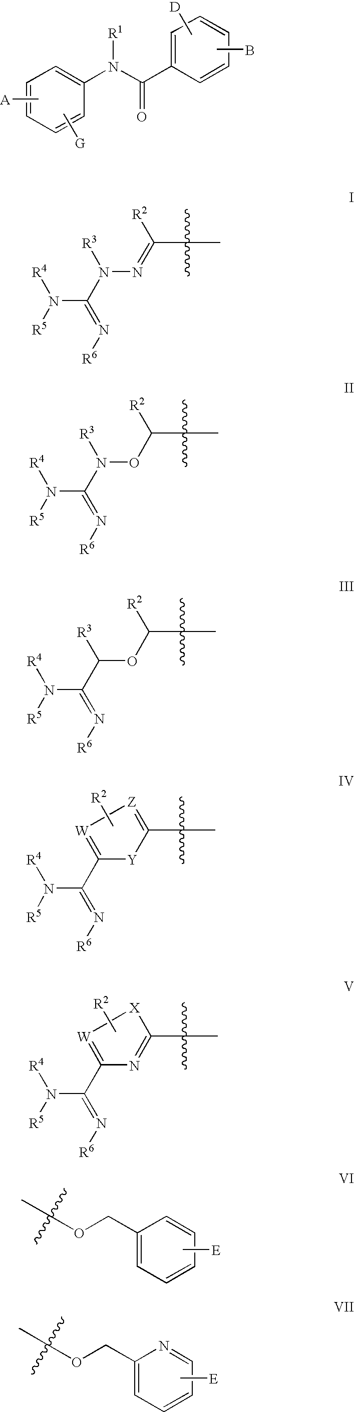 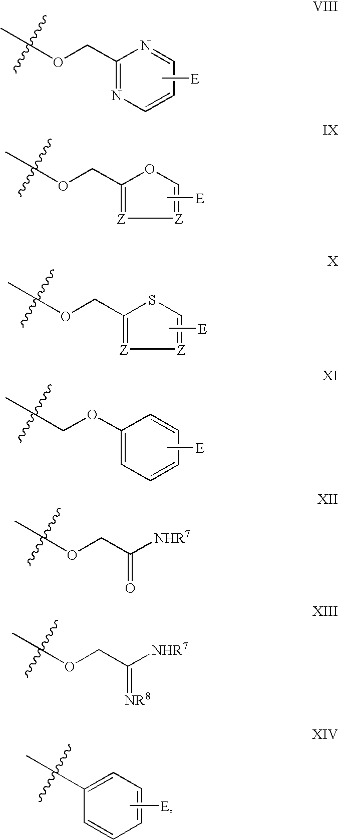
wherein A is selected from formulas I-V, and B is selected from hydrogen, halide, or formulas VI-XIV, or B is selected from formulas I-V, and A is selected from hydrogen, halide, or formulas VI-XIV; and wherein D, E, and G are independently hydrogen, alkyl, aralkyl, heteroalkyl, alkene, heteroalkene, alkyne, heteroalkyne, aryl, heteroaryl, alkoxy, hydroxy, halogen, amino, nitro, alkylamino, sulfhydryl, or alkylthio; and R¹⁻⁸ are independently hydrogen, alkyl, aryl, or aralkyl; W and Z are independently CH, C-alkyl, or N; and X and Y are independently NH, N-alkyl, O, or S.

24. The compound of claim 23 having the formula:

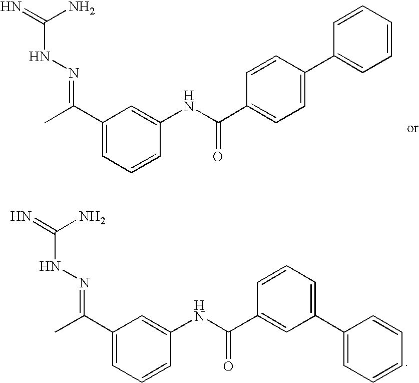

25-46. (canceled)
